SEQUENCE LISTING (14 sequences)

SEQ ID NO: 1 - RNA, 23 nt, synthetic construct
  source 1..23: mol_type = other RNA
  modified_base 1: mod_base = OTHER; note = thymine
ttatagagcaagaacactgtttt

SEQ ID NO: 2 - RNA, 21 nt, synthetic construct
  source 1..21: mol_type = other RNA
aacagtgttcttgctctataa

SEQ ID NO: 3 - DNA, 23 bp, synthetic construct
  source 1..23: mol_type = other DNA
aaaacagtgttcttgctctataa

SEQ ID NO: 4 - DNA, 19 bp, synthetic construct
  source 1..19: mol_type = other DNA
taatattacataaataaaa

SEQ ID NO: 5 - RNA, 23 nt, synthetic construct
  source 1..23: mol_type = other RNA
  modified_base 1: mod_base = OTHER; note = thymine
taaagcactttattgagtttctg

SEQ ID NO: 6 - RNA, 21 nt, synthetic construct
  source 1..21: mol_type = other RNA
gaaactcaataaagtgcttta

SEQ ID NO: 7 - DNA, 24 bp, synthetic construct
  source 1..24: mol_type = other DNA
caaaggagggacatgtatcaacac

SEQ ID NO: 8 - DNA, 20 bp, synthetic construct
  source 1..20: mol_type = other DNA
ctggcaatgtttcccagtga

SEQ ID NO: 9 - DNA, 24 bp, synthetic construct
  source 1..24: mol_type = other DNA
cccaatgggccacactgtctctgc

SEQ ID NO: 10 - DNA, 23 bp, synthetic construct
  source 1..23: mol_type = other DNA
cgtactggaagacacttggcatt

SEQ ID NO: 11 - DNA, 21 bp, synthetic construct
  source 1..21: mol_type = other DNA
gagtcgttggctgtgaaaacc

SEQ ID NO: 12 - DNA, 23 bp, synthetic construct
  source 1..23: mol_type = other DNA
cccgttccatgaattcgcggatg

SEQ ID NO: 13 - RNA, 1237 nt, Mus musculus
  source 1..1237: mol_type = mRNA
ctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggatggcttcccttcgactcttcctcctttgcctcgctggactggtatttgtgtctgaagctggccccgcgggtgctggagaatccaaatgtcctctgatggtcaaagtcctggatgctgtccgaggcagccctgctgtagacgtggctgtaaaagtgttcaaaaagacctctgagggatcctgggagccctttgcctctgggaagaccgcggagtctggagagctgcacgggctcaccacagatgagaagtttgtagaaggagtgtacagagtagaactggacaccaaatcgtactggaagacacttggcatttccccgttccatgaattcgcggatgtggttttcacagccaacgactctggccatcgccactacaccatcgcagccctgctcagcccatactcctacagcaccacggctgtcgtcagcaacccccagaattgagagactcagcccaggaggaccaggatcttgccaaagcagtagcatcccatttgtaccaaaacagtgttcttgctctataaaccgtgttagcagctcaggaagatgccgtgaagcattcttattaaaccacctgctatttcattcaaactgtgtttcttttttatttcctcatttttctcccctgctcctaaaacccaaaatcttctaaagaattctagaaggtatgcgatcaaactttttaaagaaagaaaatactttttgactcatggtttaaaggcatcctttccatcttggggaggtcatgggtgctcctggcaacttgcttgaggaagataggtcagaaagcagagtggaccaaccgttcaatgttttacaagcaaaacatacactaagcatggtctgtagctattaaaagcacacaatctgaagggctgtagatgcacagtagtgttttcccagagcatgttcaaaagccctgggttcaatcacaatactgaaaagtaggccaaaaaacattctgaaaatgaaatatttgggtttttttttataacctttagtgactaaataaagacaaatctaagagactaaaaaaaaaaaaaaaaaa

SEQ ID NO: 14 - RNA, 1971 nt, Mus musculus
  source 1..1971: mol_type = mRNA
actcctgggcaggcagcggggccatcggcagacgccatgacggctctgttgttcctggggtctctgctgatgagtctggatctgacactttcggctccaccatggaaagactccaagaaatttaaggacgcacctgatgggcccacagtggttctcactgtggatgggaggctctgccattttccctttcagtaccaccgtcagctacaccacaaatgcatccacaaaaggcggccaggctcccgcccctggtgtgctaccacccccaactttgatgaagatcagcaatggggatactgcttggagcccaagaaagtgaaagaccattgcagcaaacacaacccgtgccacaaaggagggacatgtatcaacacccccaatgggccacactgtctctgccctgaacacctcactgggaaacattgccagaaagagaaatgctttgagcctcagcttctcaagttcttccacgagaatgagctatggtttagaacggggccaggaggtgtggccaggtgcgagtgcaaaggttctgaggctcactgcaagccggtggccagccaggcctgcagcatcaatccgtgccttaatgggggcagctgcctcctcgtggaggaccacccactgtgccgttgccctacaggctacactggatatttttgcgacttggacctttgggcgacctgctatgaaggcagggggctcagctaccggggccaggctggaactacgcaatcgggtgcgccatgtcagcggtggaccgtggaggccacctaccggaacatgactgagaagcaagcgctaagctggggcctgggccaccacgcattttgccggaacccagataatgacacacgtccatggtgcttcgtctggagtggcgacaggctgagctgggactattgcggcctggagcagtgccagacgccaacgtttgcacctctagttgtccctgagagtcaggaggagtccccgtcccaggcaccatctctgtcccatgcaccaaatgactcgaccgatcatcagacttctctgtccaagaccaacacgatgggctgcggacagaggttccgcaagggactgtcctcgttcatgcgcgtggtgggcggactagtggctctgcctgggtcgcacccctacatcgctgcactgtactggggtaacaacttctgcgcgggcagtctcatcgccccctgttgggtgctgaccgcggctcactgcctgcagaatcggccagcgcccgaggaactgacagtggtacttggtcaagatcgccacaaccagagctgcgagtggtgccagactctggctgtgcgctcctaccgccttcacgagggcttctcctccatcacctaccagcacgacttggctctgctgcgcctgcaggaaagcaaaaccaacagttgcgcgatcctgtcacctcacgttcagcctgtgtgtctacccagcggcgcggccccaccctctgagacagtgctctgcgaggtggccggctggggtcaccagttcgagggggctgaagaatactccaccttcctgcaggaggcacaggttccctttatcgccctggatcgctgctccaactctaacgtgcacggagacgccattctccctgggatgctttgcgctggcttcttggagggaggcaccgatgcctgccagggtgactccgggggccctctggtgtgtgaggaaggaactgcagaacatcagctcaccctgcgcggagtcatcagctggggctccggctgtggtgaccgcaacaagcccggagtctacacagacgtggccaactacctggcttggatccagaagcatattgcttcataactaaccaggctttatccttccctccttgtgtgctccttgggatgggacgatgaatgtggcatgctgggtcacagtgaagctagtgccccgacactgggggcacagaaactcaataaagtgctttgaaaacgttcctcagaa

(19) United States
(12) Patent Application Publication
(10) Pub. No.: US 2025/0346898 A1
(43) Pub. Date: Nov. 13, 2025

(54) Linkage Modified Oligomeric Compounds and Uses Thereof

(71) Applicant: Ionis Pharmaceuticals, Inc., Carlsbad, CA (US)

(72) Inventors: Thazha P. Prakash, Carlsbad, CA (US); Mehran Nikan, Oceanside, CA (US); Qingfeng Li, San Marcos, CA (US); Eric E. Swayze, Encinitas, CA (US)

(73) Assignee: Ionis Pharmaceuticals, Inc., Carlsbad, CA (US)

(21) Appl. No.: 18/868,918

(22) PCT Filed: Jun. 1, 2023
(86) PCT No.: PCT/US2023/067788

§ 371 Date: Nov. 25, 2024

Related U.S. Application Data
(60) Provisional application No. 63/348,275, filed on Jun. 2, 2022.
(60) Provisional application No. 63/387,478, filed on Dec. 14, 2022.

(51) Int. Cl. C12N 15/113

(52) U.S. Cl. CPC C12N 15/113; C12N 2310/14; C12N 2310/312; C12N 2310/313; C12N 2310/321; C12N 2310/322; C12N 2310/351

(57) ABSTRACT

The present disclosure provides oligomeric compounds (including oligomeric compounds that are antisense agents or portions thereof) comprising a modified oligonucleotide having at least one modified internucleoside linking group.

### SEQUENCE LISTING

The present application is being filed along with a Sequence Listing in electronic format. The Sequence Listing is provided as a file entitled CHEM0107SEQ.xml created Jun. 1, 2023 which is 17 kb in size. The information in the electronic format of the sequence listing is incorporated herein by reference in its entirety.

### FIELD

The present disclosure provides RNAi agents comprising at least one modified oligonucleotide having at least one modified internucleoside linking group within the seed region.

### BACKGROUND

The principle behind antisense technology is that an antisense compound hybridizes to a target nucleic acid and modulates the amount, activity, and/or function of the target nucleic acid. For example, in certain instances, antisense compounds result in altered transcription or translation of a target. Such modulation of expression can be achieved by, for example, target RNA degradation or occupancy-based inhibition. An example of modulation of RNA target function by degradation is RNase H-based degradation of the target RNA upon hybridization with a DNA-like antisense compound.

Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Another example of modulation of gene expression is the use of antisense compounds in a CRISPR system. Regardless of the specific mechanism, sequence-specificity makes antisense compounds attractive as tools for target validation and gene functionalization, as well as therapeutics to selectively modulate the expression of genes involved in the pathogenesis of disease.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, tolerability, pharmacokinetics, or affinity for a target nucleic acid.

### SUMMARY

The present disclosure provides oligomeric compounds (including oligomeric compounds that are antisense agents or portions thereof) comprising modified oligonucleotides consisting of linked nucleosides linked through internucleoside linking groups, wherein at least one of the internucleoside linking groups is not a phosphodiester or phosphorothioate.

In certain embodiments, the internucleoside linking groups may be included in the seed region of an antisense RNAi oligonucleotide. Such internucleoside linking groups as described herein may provide seed-region destabilization of RNA interference (RISC) complexes. In certain embodiments, the internucleoside linkages described herein may increase selectivity of RNA interference when compared to an analogous RNAi oligonucleotide that includes only typical (e.g., phosphodiester or phosphorothioate) internucleoside linkages in the seed region. In certain embodiments, an RNAi agent provided herein has improved selectivity compared to an analogous agent containing only phosphodiester and phosphorothioate internucleoside linkages and lacking a destabilizing sugar moiety (e.g., an acyclic sugar moiety such as UNA or GNA) in the seed region thereof. In certain embodiments, an RNAi agent provided herein has improved selectivity compared to an analogous agent containing a destabilizing sugar moiety (e.g., an acyclic sugar moiety such as UNA or GNA). In certain embodiments, an RNAi agent provided herein has improved on-target potency compared to an analogous agent containing a destabilizing sugar moiety (e.g., an acyclic sugar moiety such as UNA or GNA).

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

Herein, compounds described as having “the nucleobase sequence of” a SEQ ID, unless otherwise indicated, include such compounds wherein each nucleobase is independently modified or unmodified, independent of nucleobase modifications, or absence of nucleobase modifications, indicated in the refenced SEQ ID. Further, such description of compounds by reference to a SEQ ID does not limit sugar or internucleoside linkage modifications, which, unless otherwise indicated, are independent of nucleobase sequence and nucleobase modification.

It is understood that the nucleobase sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH(H) sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, a modified oligonucleotide having the nucleobase sequence “ATCGATCG” encompasses any modified oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and modified oligonucleotides having other modified nucleobases, such as “AT^{m}CGAUCG,” wherein ^{m}C indicates a cytosine base comprising a methyl group at the 5-position. One of skill in the art will readily appreciate that labeling such nucleic acid compounds “RNA” or “DNA” does not alter or limit such nucleic acid compounds. While effort has been made to accurately describe compounds in the accompanying ST.26 compliant sequence listing, should there be any discrepancies between a description in this specification and in the accompanying sequence listing, the description in the specification controls.

As used herein, “2′-substituted” in reference to a furanosyl sugar moiety or nucleoside comprising a furanosyl sugar moiety means the furanosyl sugar moiety or nucleoside comprising the furanosyl sugar moiety comprises a substituent other than H or OH at the 2′-position and is a non-bicyclic furanosyl sugar moiety. 2′-substituted furanosyl sugar moieties do not comprise additional substituents at other positions of the furanosyl sugar moiety other than a nucleobase and/or internucleoside linkage(s) when in the context of an oligonucleotide.

As used herein, “4′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 4′-position and is a non-bicyclic furanosyl sugar moiety. 4′-substituted furanosyl sugar moieties do not comprise additional substituents at other positions of the furanosyl sugar moiety other than a nucleobase and/or internucleoside linkage(s) when in the context of an oligonucleotide.

As used herein, “5′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 5′-position and is a non-bicyclic furanosyl sugar moiety. 5′-substituted furanosyl sugar moieties do not comprise additional substituents at other positions of the furanosyl sugar moiety other than a nucleobase and/or internucleoside linkage(s) when in the context of an oligonucleotide.

As used herein, “administration” or “administering” refers to routes of introducing a compound or composition provided herein to a subject to perform its intended function. Examples of routes of administration that can be used include, but are not limited to, administration by inhalation, subcutaneous injection, intrathecal injection, and oral administration.

As used herein, “antisense activity” means any detectable and/or measurable change attributable to the hybridization of an antisense oligonucleotide to its target nucleic acid. In certain embodiments, antisense activity is a decrease in the amount or expression of a target nucleic acid or protein encoded by such target nucleic acid compared to target nucleic acid levels or target protein levels in the absence of the antisense oligonucleotide.

As used herein, “antisense agent” means an antisense oligonucleotide or an oligonucleotide duplex comprising an antisense oligonucleotide.

As used herein, “antisense compound” means an antisense oligonucleotide or an oligonucleotide duplex comprising an antisense oligonucleotide.

As used herein, “antisense oligonucleotide” means an oligonucleotide that is complementary to a target nucleic acid and is capable of achieving at least one antisense activity. Antisense oligonucleotides include but are not limited to RNAi antisense modified oligonucleotides and RNase H antisense modified oligonucleotides. In certain embodiments, an antisense oligonucleotide is paired with a sense oligonucleotide to form an oligonucleotide duplex. In certain embodiments, an antisense oligonucleotide is unpaired and is a single-stranded antisense oligonucleotide. In certain embodiments, an antisense oligonucleotide comprises a conjugate group.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified bicyclic furanosyl sugar moiety. In certain embodiments, the bicyclic sugar moiety does not comprise a furanosyl moiety.

As used herein, “cEt” or “constrained ethyl” or “cEt sugar moiety” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the methyl group of the bridge is in the S configuration. A cEt bicyclic sugar moiety is in the β-D configuration.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms consisting of a conjugate moiety and a conjugate linker.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “conjugate linker” means a group of atoms comprising at least one bond.

As used herein, “cytotoxic” or “cytotoxicity” in the context of an effect of an oligomeric compound or a parent oligomeric compound on cultured cells means an at least 2-fold increase in caspase activation following administration of 10 μM or less of the oligomeric compound or parent oligomeric compound to the cultured cells relative to cells cultured under the same conditions but that are not administered the oligomeric compound or parent oligomeric compound. In certain embodiments, cytotoxicity is measured using a standard in vitro cytotoxicity assay.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “hepatotoxic” in the context of a mouse means a plasma ALT level that is above 300 units per liter. Hepatotoxicity of an oligomeric compound or parent oligomeric compound that is administered to a mouse is determined by measuring the plasma ALT level of the mouse 24 hours to 2 weeks following at least one dose of 1-150 mg/kg of the compound.

As used herein, “hepatotoxic” in the context of a human means a plasma ALT level that is above 150 units per liter. Hepatotoxicity of an oligomeric compound or parent oligomeric compound that is administered to a human is determined by measuring the plasma ALT level of the human 24 hours to 2 weeks following at least one dose of 10-300 mg of the compound.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression or activity in an untreated or control sample and does not necessarily indicate a total elimination of expression or activity.

As used herein, “internucleoside linkage” or “internucleoside linking group” means a group or bond that forms a covalent linkage between adjacent nucleosides in an oligonucleotide. As used herein “modified internucleoside linkage” means any internucleoside linkage other than a naturally occurring, phosphodiester internucleoside linkage. “Phosphorothioate linkage” means a modified internucleoside linkage in which one of the non-bridging oxygen atoms of a phosphodiester is replaced with a sulfur atom. Modified internucleoside linkages may or may not contain a phosphorus atom. A “neutral internucleoside linkage” is a modified internucleoside linkage that does not have a negatively charged phosphate in a buffered aqueous solution at pH=7.0. A modified internucleoside linkage may optionally comprise a conjugate group.

As used herein, “linked nucleosides” are nucleosides that are connected in a continuous sequence (i.e. no additional nucleosides are present between those that are linked).

As used herein, “maximum tolerated dose” means the highest dose of a compound that does not cause unacceptable side effects. In certain embodiments, the maximum tolerated dose is the highest dose of a modified oligonucleotide that does not cause an ALT elevation of three times the upper limit of normal as measured by a standard assay.

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulating” refers to changing or adjusting a feature in a cell, tissue, organ or organism.

As used herein, “2′-deoxynucleoside” means a nucleoside according to the structure:

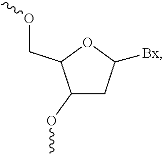

wherein Bx is a nucleobase.

As used herein, “2′-deoxy sugar moiety” means the sugar moiety of a 2′-deoxynucleoside. As indicated in the above structure, a 2′-deoxy sugar moiety can have any stereochemistry. For example, 2′-deoxy sugar moieties include, but are not limited to 2′-β-D-deoxyribosyl sugar moieties and 2′-β-D-deoxyxylosyl sugar moieties.

As used herein, “2′-β-D-deoxyribosyl nucleoside” means a nucleoside according to the structure:

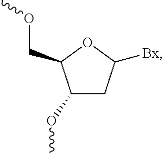

wherein Bx is a nucleobase.

As used herein, “2′-β-D-deoxyribosyl sugar moiety” means the sugar moiety of a 2′-β-D-deoxyribosyl nucleoside. The nucleobase of a 2′-deoxynucleoside or 2′-β-D-deoxyribosyl nucleoside may be a modified nucleobase or any natural nucleobase, including but not limited to an RNA nucleobase (uracil).

As used herein, “2′-MOE nucleoside” means a nucleoside according to the structure:

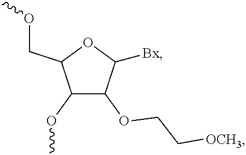

wherein Bx is a nucleobase.

As used herein, “2′-MOE sugar moiety” means the sugar moiety of a 2′-MOE nucleoside as defined herein.

As used herein, “ribo-2′-MOE nucleoside” means a nucleoside according to the structure:

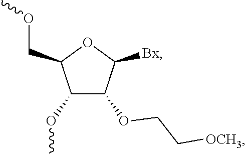

wherein Bx is a nucleobase.

As used herein, “Ribo-2′-MOE sugar moiety” means the sugar moiety of a ribo-2′-MOE nucleoside as defined herein.

“MOE” means an —OCH₂CH₂OCH₃ group.

As used herein, “2′-OMe nucleoside” means a nucleoside according to the structure:

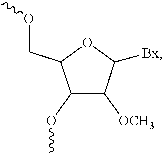

wherein Bx is a nucleobase.

As used herein, “2′-OMe sugar moiety” means the sugar moiety of a 2′-OMe nucleoside. As indicated in the above structure, a 2′-OMe sugar moiety can have any stereochemistry. For example, 2′-OMe sugar moieties include, but are not limited to 2′-OCH₃-β-D-xylosyl sugar moieties, 2′-OCH₃-α-L-ribosyl sugar moieties, and ribo-2′-OMe sugar moieties as defined herein.

As used herein, “ribo-2′-OMe nucleoside” means a nucleoside according to the structure:

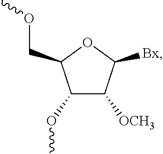

wherein Bx is a nucleobase.

As used herein, “ribo-2′-OMe sugar moiety” means the sugar moiety of a ribo-2′-OMe nucleoside.

“2′-F nucleoside” means a nucleoside according to the structure:

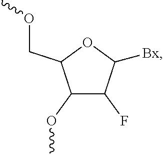

wherein Bx is a nucleobase.

As used herein, “2′-F sugar moiety” means the sugar moiety of a 2′-F nucleoside. As indicated in the above structure, a 2′-F sugar moiety can have any stereochemistry. For example, 2′-F sugar moieties include, but are not limited to, 2′-F-β-D-xylosyl sugar moieties, 2′-F-D-D-arabinosyl sugar moieties, 2′-F-α-L-ribosyl sugar moieties, and ribo-2′-F sugar moieties as defined herein.

As used herein, “ribo-2′-F nucleoside” means a nucleoside according to the structure:

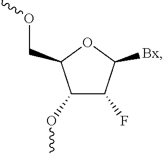

wherein Bx is a nucleobase.

As used herein, “ribo-2′-F sugar moiety” means the sugar moiety of a ribo-2′-F nucleoside as defined herein.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a modified nucleobase is a group of atoms capable of pairing with at least one unmodified nucleobase. A universal base is a nucleobase that can pair with any one of the five unmodified nucleobases. 5-methylcytosine (^{m}C) is one example of a modified nucleobase.

As used herein, “nucleobase sequence” means the order of contiguous nucleobases in a nucleic acid or oligonucleotide independent of any sugar moiety or internucleoside linkage modification. Unless otherwise specified, uracil nucleobases are interchangeable with thymine (and vice versa), and cytosine nucleobases are interchangeable with 5-methylcytosine (and vice versa).

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. A modified nucleoside may comprise a conjugate group.

As used herein, “oligomeric compound” means a compound consisting of (1) an oligonucleotide (a single-stranded oligomeric compound) or two oligonucleotides hybridized to one another (a double-stranded oligomeric compound); and (2) optionally one or more additional features, such as a conjugate group or terminal group which may be attached to the oligonucleotide of a single-stranded oligomeric compound or to one or both oligonucleotides of a double-stranded oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 12-80 linked nucleosides, and optionally a conjugate group or terminal group. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, liquids, powders, or suspensions that can be aerosolized or otherwise dispersed for inhalation by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds (including oligomeric compounds that are antisense agents or portions thereof), i.e., salts that retain the desired biological activity of the compound and do not impart undesired toxicological effects thereto.

As used herein “pharmaceutical composition” means a mixture of substances suitable for administering to a subject. For example, a pharmaceutical composition may comprise an antisense compound and an aqueous solution.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNA (ssRNA), and microRNA, including microRNA mimics. RNAi agents may comprise conjugate groups and/or terminal groups. In certain embodiments, an RNAi agent modulates the amount, activity, and/or splicing of a target nucleic acid. The term RNAi agent excludes antisense agents that act through RNase H.

As used herein, “RNAi oligonucleotide” means an RNAi antisense modified oligonucleotide or a RNAi sense modified oligonucleotide.

As used herein, “antisense RNAi oligonucleotide” means an oligonucleotide comprising a region that is complementary to a target sequence, and which includes at least one chemical modification suitable for RNAi.

As used herein, “antisense RNAi oligomeric compound” means a single-stranded oligomeric compound comprising a region that is complementary to a target sequence, and which includes at least one chemical modification suitable for RNAi.

As used herein, “sense RNAi oligonucleotide” means an oligonucleotide comprising a region that is complementary to a region of an RNAi antisense modified oligonucleotide, and which is capable of forming a duplex with such RNAi antisense modified oligonucleotide.

As used herein, “sense RNAi oligomeric compound” means a single-stranded oligomeric compound comprising a region that is complementary to a region of an RNAi antisense modified oligonucleotide and/or an RNAi antisense oligomeric compound, and which is capable of forming a duplex with such RNAi antisense modified oligonucleotide and/or RNAi antisense oligomeric compound.

A duplex formed by an antisense RNAi oligonucleotide and/or an antisense RNAi oligomeric compound with a sense RNAi oligonucleotide and/or a sense RNAi oligomeric compound is referred to as a double-stranded RNAi agent (dsRNAi) or a short interfering RNA (siRNA) or an RNAi duplex.

As used herein, the term “seed region” in reference to an antisense RNAi oligonucleotide refers to a region at or near the 5′end of an antisense RNAi oligonucleotide having a nucleobase sequence that is important for target nucleic acid recognition by the antisense RNAi oligonucleotide. In certain embodiments, a seed region comprises nucleobases 2-8, nucleobases 2-7, nucleobases 1-7, nucleobases 1-6, nucleobases 1-7, or nucleobases 1-8 of an antisense RNAi oligonucleotide, counting from the 5′-end.

As used herein, the term “single-stranded” in reference to an oligomeric compound means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “stabilized phosphate group” refers to a 5′-chemical moiety that results in stabilization of a 5′-phosphate moiety of the 5′-terminal nucleoside of an oligonucleotide, relative to the stability of an unmodified 5′-phosphate of an unmodified nucleoside under biologic conditions. Such stabilization of a 5′-phophate group includes but is not limited to resistance to removal by phosphatases. Stabilized phosphate groups include, but are not limited to, 5′-vinyl phosphonates, 5′-methyl phosphonates, and 5′-cyclopropyl phosphonate.

As used herein, “stereorandom” or “stereorandom chiral center” in the context of a population of molecules of identical molecular formula means a chiral center that is not controlled during synthesis, or enriched following synthesis, for a particular absolute stereochemical configuration. The stereochemical configuration of a chiral center is random when it is the result of a synthetic method that is not designed to control the stereochemical configuration. For example, in a population of molecules comprising a stereorandom chiral center, the number of molecules having the (S) configuration of the stereorandom chiral center may be the same as the number of molecules having the (R) configuration of the stereorandom chiral center (“racemic”). In certain embodiments, the stereorandom chiral center is not racemic because one absolute configuration predominates following synthesis, e.g., due to the action of non-chiral reagents near the enriched stereochemistry of an adjacent sugar moiety. In certain embodiments, the stereorandom chiral center is at the phosphorous atom of one or more of a stereorandom phosphorothioate internucleoside linkage, a mesyl phosphoramidate internucleoside linkage, an internucleoside linkage of a region of Formula I, or an internucleoside linkage of Formula II.

As used herein, “subject” means a human or non-human animal selected for treatment or therapy.

As used herein, “sugar moiety” means an unmodified sugar moiety or a modified sugar moiety. As used herein, “unmodified sugar moiety” means a β-D-ribosyl moiety, as found in naturally occurring RNA, or a 2′-β-D-deoxyribosyl sugar moiety as found in naturally occurring DNA. As used herein, “modified sugar moiety” or “modified sugar” means a sugar surrogate or a furanosyl sugar moiety other than a β-D-ribosyl or a 2′-β-D-deoxyribosyl. Modified furanosyl sugar moieties may be modified or substituted at a certain position(s) of the sugar moiety, or unsubstituted, and they may or may not be stereo-non-standard sugar moieties. Modified furanosyl sugar moieties include bicyclic sugars and non-bicyclic sugars. As used herein, “sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid,” “target RNA,” “target RNA transcript” and “nucleic acid target” means a nucleic acid that an oligomeric compound, such as an antisense compound, is designed to affect. In certain embodiments, an oligomeric compound comprises an oligonucleotide having a nucleobase sequence that is complementary to more than one RNA, only one of which is the target RNA of the oligomeric compound. In certain embodiments, the target RNA is an RNA present in the species to which an oligomeric compound is administered.

As used herein, “therapeutic index” means a comparison of the amount of a compound that causes a therapeutic effect to the amount that causes toxicity. Compounds having a high therapeutic index have strong efficacy and low toxicity. In certain embodiments, increasing the therapeutic index of a compound increases the amount of the compound that can be safely administered.

As used herein, “treat” refers to administering a compound or pharmaceutical composition to an animal in order to effect an alteration or improvement of a disease, disorder, or condition in the animal.

As used herein, “translation suppression element,” means any sequence and/or secondary structure in the 5′-UTR of a target transcript that reduces, inhibits, and/or suppresses translation of the target transcript. In certain embodiments, a translation suppression element comprises a uORF. In certain embodiments, a translation suppression element does not comprise a uORF. In certain embodiments, a translation suppression element comprises one or more stem-loops. In certain embodiments, a translation suppression element comprises greater than 60%, greater than 70%, or greater than 80% GC content. In certain embodiments, the translation suppression element is a uORF. In certain embodiments, the translation suppression element is a stem-loop.

As used herein, “alkyl” refers to a saturated straight or branched hydrocarbon substituent group containing up to twenty four carbon atoms. Examples of alkyl groups include without limitation, methyl, ethyl, propyl, butyl, isopropyl, n-hexyl, octyl, decyl, dodecyl and the like. Alkyl groups typically include from 1 to 20 carbon atoms (“C₁-C₂₀ alkyl”), more typically from 1 to 12 carbon atoms (“C₁-C₁₂ alkyl”) with from 1 to 6 carbon atoms (“C₁-C₆ alkyl”) being more preferred. Alkyl groups as used herein may optionally include one or more further substituent groups.

As used herein, “alkenyl,” refers to a straight or branched hydrocarbon chain substituent group containing up to twenty four carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to 20 carbon atoms, more typically from 2 to 12 carbon atoms with from 2 to 6 carbon atoms being more preferred. Alkenyl groups as used herein may optionally include one or more further substituent groups.

As used herein, “alkynyl”, refers to a straight or branched hydrocarbon substituent group containing up to twenty four carbon atoms and having at least one carbon-carbon triple bond. Examples of alkynyl groups include, without limitation, ethynyl, 1-propynyl, 1-butynyl, and the like. Alkynyl groups typically include from 2 to 20 carbon atoms, more typically from 2 to 12 carbon atoms with from 2 to 6 carbon atoms being more preferred. Alkynyl groups as used herein may optionally include one or more further substituent groups.

As used herein, “alkoxy” refers to an alkyl-O-substituent group, where alkyl is as defined herein. Examples of alkoxy groups include without limitation, methoxy, ethoxy, propoxy, isopropoxy, n-butoxy, see-butoxy, tert-butoxy, n-pentoxy, neopentoxy, n-hexoxy and the like. Alkoxy groups as used herein may optionally include further substituent groups.

As used herein, “aryl” refers to a carbocyclic ring system substituent group having one or more aromatic rings. The aryl may be monocyclic or may include two or more fused rings. Examples of aryl groups include without limitation, phenyl, naphthyl, tetrahydronaphthyl, indanyl, idenyl and the like. Preferred aryl ring systems have from 6 to 10 ring atoms. Aryl groups as used herein may optionally include further substituent groups.

As used herein, “cycloalkyl” refers to a saturated or unsaturated carbocyclic ring system substituent group that does not include an aromatic ring. The cycloalkyl may be monocyclic or may include two or more fused rings. Examples of cycloalkyl groups include without limitation, cyclopropyl, cyclobutyl, cyclopentyl, cyclohexyl, cyclohexenyl, and the like. Preferred cycloalkyl ring systems have from 3 to 10 ring atoms (“C₃-C₁₀ cycloalkyl”). Cycloalkyl groups as used herein may optionally include further substituent groups.z

As used herein, “halo” or “halogen” refers to a substituent group selected from fluoride, chloride, bromide and iodide.

As used herein, “heteroaryl” refers to a substituent group comprising a ring system in which at least one of the rings is aromatic, and at least one ring includes one or more ring heteroatoms. The heteroaryl may be monocyclic or may include two or more fused rings. Heteroaryl groups include at least one ring atom selected from sulfur, nitrogen or oxygen, wherein the sulfur is optionally present as a sulfoxide or sulfone, and wherein the nitrogen is optionally present as an N-oxide. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, thiophenyl, furanyl, quinolinyl, and the like. Heteroaryl groups as used herein may optionally include further substituent groups.

As used herein, “heteroalkyl” refers to an alkyl substituent group as defined herein in which one or more CH₂ units are replaced with a heteroatom independently selected from O, NH, N(C₁₋₆alkyl), S, SO, and SO₂, except that heteroalkyl does not encompass groups defined herein as alkoxy. Examples of heteroalkyl groups include without limitation, methoxypropyl, ethoxymethyl, propylsulfonyl, 1-(methylthio)propan-2-yl, methyl(methylthio)amino, N-propylamino, 2-(methylamino)ethyl, and the like. Heteroalkyl groups typically include from 1 to 20 carbon atoms (“C₁-C₂₀ heteroalkyl”), more typically from 1 to 12 carbon atoms (“C₁-C₁₂ heteroalkyl”) with from 1 to 6 carbon atoms (“C₁-C₆ heteroalkyl”) being more preferred. Heteroalkyl groups as used herein may optionally include one or more further substituent groups.

As used herein, “heterocyclyl” refers to a substituent group comprising a ring system in which none of the rings are aromatic, and at least one ring includes one or more ring heteroatoms. Heterocyclyl is also meant to include fused ring systems including systems where one or more of the fused rings contain no heteroatoms. Heterocyclyl groups include at least one ring atom selected from sulfur, nitrogen or oxygen, wherein the sulfur is optionally present as a sulfoxide or sulfone. Examples of heterocyclyl groups include without limitation, morpholino, oxirane, tetrahydropyranyl, tetrahydrothienyl, sulfolanyl, and the like. Heterocyclyl groups as used herein may optionally include further substituent groups.

### Certain Embodiments

The present disclosure provides the following non-limiting embodiments:

Embodiment 1. An RNAi agent comprising an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, wherein the antisense RNAi oligonucleotide comprises at least one region having Formula I:

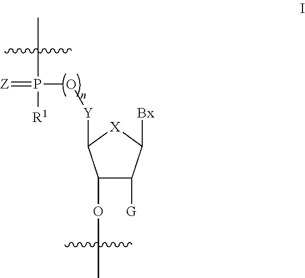

wherein
X is O or O—CH₂;
Z is O or S;
R¹ is selected from OH, C₁-C₂₀ alkyl, C₁-C₂₀ heteroalkyl, C₃-C₁₀ cycloalkyl, C₆-C₁₀ aryl, 3-10 membered heterocyclyl, and 3-10 membered heteroaryl, and wherein R¹ is optionally substituted with C₁-C₂₀ alkyl, C₁-C₂₀ heteroalkyl, C₃-C₁₀ cycloalkyl, C₆-C₁₀ aryl, 3-10 membered heterocyclyl, 3-10 membered heteroaryl, or R^{A}COOR^{D}, wherein R^{A} is C₁-C₆ alkyl and R^{D} is H or C₁-C₆ alkyl;
n is 0 or 1;
Y is C₁-C₆ alkyl, C₃-C₁₀ cycloalkyl, or 3-10 membered heterocyclyl; provided that if n is 1 and Y is CH₂ or CH₂(CH₃), then R¹ is not OH;
and
each G is independently H, halogen, OH, or O[C(R⁴)(R⁵)]ₚ—[(C═O)_{q}—R⁶]ⱼ—R⁷; wherein
   each R⁴ and R⁵ is, independently, H, halogen, C₁-C₆ alkyl or substituted C₁-C₆ alkyl;
   each R⁶ is O, S or N(E¹);
   R⁷ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, or N(E²)(E³);
   E¹, E² and E³ are each, independently, H, C₁-C₆ alkyl, or substituted C₁-C₆ alkyl;
   p is from 1 to 6;
   q is 0 or 1;
   j is 0 or 1;
   each substituted group comprises one or more optionally protected substituent groups independently selected from halogen, OJ¹, N(J¹)(J²), =NJ¹, SJ¹, N₃, CN, OC(=Q)J¹, OC(=Q)-N(J¹)(J²), and C(=Q)N(J¹)(J²);
      Q is O, S or NJ³;
      J¹, J² and J³ are each, independently, H or C₁-C₆ alkyl;
Bx is a heterocyclic base moiety.

Embodiment 2. The RNAi agent of claim 1, wherein X is O.

Embodiment 3. The RNAi agent of claim 1, wherein X is O—CH₂.

Embodiment 4. The RNAi agent of any of claims 1-3, wherein Z is O.

Embodiment 5. The RNAi agent of any of claims 1-3, wherein Z is S.

Embodiment 6. The RNAi agent of any of claims 1-5, wherein n is 1.

Embodiment 7. The RNAi agent of any of claims 1-6, wherein Y is CH₂.

Embodiment 8. The RNAi agent of any of claims 1-7, wherein R¹ is —[C(R⁸)(H)]ₘO[C(R⁹)(H)]ₗ—R¹⁰, wherein
each R⁸ and R⁹ is independently selected from H or C₁-C₃ alkyl;
R¹⁰ is H or halogen;
m is from 1 to 6;
and t is from 1 to 6.

Embodiment 9. The RNAi agent of claim 8, wherein each R⁸ is H, and m is from 1-3.

Embodiment 10. The RNAi agent of claim 9, wherein each R⁹ is H and t is from 1-3.

Embodiment 11. The RNAi agent of claim 8, wherein R¹ is methoxypropyl.

Embodiment 12. The RNAi agent of any of claims 1-7, wherein R¹ is C₁-C₆ alkyl.

Embodiment 13. The RNAi agent of claim 12, wherein R¹ is propyl or isopropyl.

Embodiment 14. The RNAi agent of any of claims 1-5, wherein n is 0 and Y is cyclopropyl.

Embodiment 15. The RNAi agent of any of claims 1-14, wherein G is selected from OMe, F, or H.

Embodiment 16. The RNAi agent of claim 15, wherein G is F.

Embodiment 17. The RNAi agent of claim 15, wherein G is OMe.

Embodiment 18. The RNAi agent of any of claims 1-17, having exactly one region having Formula I.

Embodiment 19. The RNAi agent of claim 17, wherein the region having Formula I is within the first 8 nucleosides of the antisense RNAi oligonucleotide.

Embodiment 20. The RNAi agent of claim 18, wherein the region having Formula I includes the 6^{th} nucleoside from the 5′ end of the antisense RNAi oligonucleotide.

Embodiment 21. The RNAi agent of claim 18, wherein the region having Formula I includes the 7^{th} nucleoside from the 5′ end of the antisense RNAi oligonucleotide.

Embodiment 22. An RNAi agent, comprising an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, and wherein the antisense RNAi oligonucleotide contains at least one internucleoside linkage having Formula II:

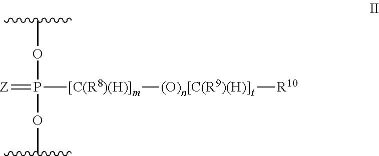

wherein Z is O or S;
   each R⁸ is independently selected from H or C₁-C₃ alkyl;
   each R⁹ is absent or independently selected from H, C₁-C₃ alkyl;
   R¹⁰ is H, halogen, or COOR^{D}, wherein R^{D} is H or C₁-C₃ alkyl;
   n is 0 or 1;
   m is from 1 to 6;
and t is from 0 to 6.
   Embodiment 23. The RNAi agent of claim 22, wherein Z is O.

Embodiment 24. The RNAi agent of claim 22 or 23, wherein n is 1.

Embodiment 25. The RNAi agent of any of claims 22-24, wherein R⁸, R⁹, and R¹⁰ are each H.

Embodiment 26. The RNAi agent of any of claims 22-25, wherein m is 3.

Embodiment 27. The RNAi agent of any of claims 22-26, wherein t is 1.

Embodiment 28. The RNAi agent of any of claims 22-26, wherein n is 1; R⁸, R⁹, and R¹⁰ are each H, m is 3, and t is 1.

Embodiment 29. The RNAi agent of claim 22 or 23, wherein n is 0.

Embodiment 30. The RNAi agent of claim 29, wherein R⁸, R⁹, and R¹⁰ are each H.

Embodiment 31. The RNAi agent of claim 29 or 30, wherein m+t equals 3.

Embodiment 32. The RNAi agent of claim 29, wherein R⁸ is H; m is 1; t is 1; and each of R⁹ and R¹⁰ are methyl.

Embodiment 33. The RNAi agent of claim 29, wherein m is 1, t is 0, R⁸ is H and R¹⁰ is COOR^{D} Embodiment 34. The RNAi agent of claim 33, wherein R^{D} is H.

Embodiment 35. The RNAi agent of any of claims 22-34, wherein the internucleoside linkage having Formula II is within the seed region of the antisense RNAi oligonucleotide.

Embodiment 36. The RNAi agent of any of claims 22-35, wherein the internucleoside linkage having Formula II is between nucleosides 5 to 6 or nucleosides 6 to 7 of the antisense RNAi oligonucleotide, counting from the 5′-end.

Embodiment 37. The RNAi agent of claim 36, wherein the internucleoside linkage having Formula II is between nucleosides 6 and 7 of the antisense RNAi oligonucleotide, counting from the 5′ end.

Embodiment 38. The RNAi agent of any of claims 1-37, wherein the antisense RNAi oligonucleotide consists of 21 linked nucleosides.

Embodiment 39. The RNAi agent of any of claims 1-37, wherein the antisense RNAi oligonucleotide consists of 23 linked nucleosides.

Embodiment 40. The RNAi agent of any of claims 1-39, wherein the sense RNAi oligonucleotide consists of 19 linked nucleosides.

Embodiment 41. The RNAi agent of any of claims 1-39, wherein the sense RNAi oligonucleotide consists of 21 linked nucleosides.

Embodiment 42. The RNAi agent of any of claims 1-41, wherein the antisense RNAi oligonucleotide comprises a 5′-stabilized phosphate moiety.

Embodiment 43. The RNAi agent of claim 42, wherein the stabilized phosphate moiety is a 5′-vinyl phosphonate, a 5′-methylene phosphonate, or a 5′-cyclopropyl phosphonate.

Embodiment 44. The RNAi agent of any of claims 1-43, wherein the antisense RNAi oligonucleotide does not comprise a GNA or a UNA nucleoside.

Embodiment 45. The RNAi agent of any of claims 1-43, wherein each nucleoside of the antisense RNAi oligonucleotide comprises a sugar moiety selected from 2′-fluororibosyl, 2′-O-methyl, 2′-deoxyribosyl, 2′-O-methoxyethyl, or an FHNA sugar surrogate.

Embodiment 46. The RNAi agent of any of claims 1-45, wherein the 5′-nucleoside of the antisense RNAi oligonucleotide comprises a 5′-vinylphosphonate-2′-O-methoxyethyl-β-D-ribosyl sugar moiety.

Embodiment 47. The RNAi agent of any of claims 22-36, wherein the nucleoside to the 5′ of the internucleoside linkage having Formula II comprises a 2′-β-D-deoxyribosyl sugar moiety.

Embodiment 48. The RNAi agent of any of claims 1-21, wherein each internucleoside linkage of the antisense RNAi oligonucleotide other than the region having Formula I is selected from a phosphorothioate internucleoside linkage and a phosphodiester internucleoside linkage.

Embodiment 49. The RNAi agent of any of claims 1-21, wherein each internucleoside linkage of the antisense RNAi oligonucleotide other than the region having Formula I is selected from a phosphorothioate internucleoside linkage, a phosphodiester internucleoside linkage, and a mesyl phosphoramidate internucleoside linkage.

Embodiment 50. The RNAi agent of any of claims 22-47, wherein each internucleoside linkage of the antisense RNAi oligonucleotide that does not have Formula II is selected from a phosphorothioate internucleoside linkage and a phosphodiester internucleoside linkage.

Embodiment 51. The RNAi agent of any of claims 22-47, wherein each internucleoside linkage of the antisense RNAi oligonucleotide that does not have Formula II is selected from a phosphorothioate internucleoside linkage and a phosphodiester internucleoside linkage, and a mesyl phosphoramidate internucleoside linkage.

Embodiment 52. The RNAi agent of any of claims 22-47, wherein the antisense RNAi oligonucleotide consists of 23 linked nucleosides and has an internucleoside linkage motif selected from ssooqoooooooooooooooss or ssoooqooooooooooooooss, wherein each “s” represents a phosphorothioate internucleoside linkage, each “o” represents a phosphodiester internucleoside linkage, and each “q” represents an internucleoside linkage of Formula II.

Embodiment 53. The RNAi agent of claim 52, wherein each “q” represents a methoxypropyl internucleoside linkage.

Embodiment 54. A pharmaceutical composition comprising the RNAi agent of any of claims 1-53 and a pharmaceutically acceptable carrier or diluent.

Embodiment 55. A method comprising contacting a cell with the RNAi agent or pharmaceutical composition of any of claims 1-53.

Embodiment 56. A method of modulating the amount or activity of a target nucleic acid in a cell, comprising contacting the cell with the RNAi agent or pharmaceutical composition of any of claims 1-53 and thereby modulating the amount or activity of the target nucleic acid.

Embodiment 57. A method of modulating the amount or activity of a target nucleic acid in a cell, comprising contacting the cell with the RNAi agent or pharmaceutical composition of any of claims 1-53.

Embodiment 58. The method of claims 55-57, wherein the amount or activity of a target nucleic acid is reduced.

Embodiment 59. Use of the RNAi agent or composition of any of claims 1-53 for treatment of a disease or condition.

Embodiment 60. Use of the RNAi agent or composition of any of claims 1-53 for a preparation of a medicament for treatment of a disease or condition.

Embodiment 61. An RNAi agent comprising an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, wherein the antisense RNAi oligonucleotide comprises at least one region having Formula I:

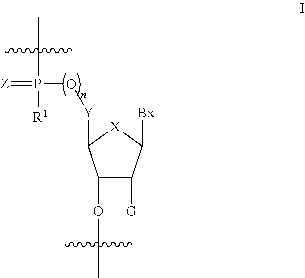

wherein
X is O or O—CH₂;
Z is O or S,
R¹ is selected from OH, C₁-C₂₀ alkyl, C₁-C₂₀ heteroalkyl, C₃-C₁₀ cycloalkyl, C₆-C₁₀ aryl, 3-10 membered heterocyclyl, and 3-10 membered heteroaryl, and wherein R¹ is optionally substituted with C₁-C₂₀ alkyl, C₁-C₂₀ heteroalkyl, C₃-C₁₀ cycloalkyl, C₆-C₁₀ aryl, 3-10 membered heterocyclyl, 3-10 membered heteroaryl, OR^{D}, NR^{D}R^{D}, SR^{D}, or R^{A}COOR^{D}, wherein R^{A} is C₁-C₆ alkyl and each R^{D} is independently H or C₁-C₆ alkyl;
n is 0 or 1;
Y is C₁-C₆ alkyl, C₃-C₁₀ cycloalkyl, or 3-10 membered heterocyclyl; provided that if n is 1 and Y is CH₂ or CH₂(CH₃), then R¹ is not OH;
and
each G is independently H, halogen, OH, or O[C(R⁴)(R⁵)]ₚ—[(C═O)_{q}—R⁶]ⱼ—R⁷; wherein
   each R⁴ and R⁵ is, independently, H, halogen, C₁-C₆ alkyl or substituted C₁-C₆ alkyl;
   each R⁶ is O, S or N(E¹);
   R⁷ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, or N(E²)(E³);
   E¹, E² and E³ are each, independently, H, C₁-C₆ alkyl, or substituted C₁-C₆ alkyl;
   p is from 1 to 6;
   q is 0 or 1;
   j is 0 or 1;
   each substituted group comprises one or more optionally protected substituent groups independently selected from halogen, OJ¹, N(J¹)(J²), =NJ¹, SJ¹, N₃, CN, OC(=Q)J¹, OC(=Q)-N(J¹)(J²), and C(=Q)N(J¹)(J²);
      Q is O, S or NJ³;
      J¹, J² and JP are each, independently, H or C₁-C₆ alkyl; and
Bx is a heterocyclic base moiety.

Embodiment 62. The RNAi agent of embodiment 61, wherein X is O.

Embodiment 63. The RNAi agent of embodiment 61, wherein X is O—CH₂.

Embodiment 64. The RNAi agent of any of embodiments 61-63, wherein Z is O.

Embodiment 65. The RNAi agent of any of embodiments 61-63, wherein Z is S.

Embodiment 66. The RNAi agent of any of embodiments 61-65, wherein n is 1.

Embodiment 67. The RNAi agent of any of embodiments 61-66, wherein Y is CH₂.

Embodiment 68. The RNAi agent of any of embodiments 61-67, wherein R¹ is —[C(R⁸)(H)]ₘO[C(R⁹)(H)]ₜ—R¹⁰, wherein
each R⁸ and R⁹ is independently selected from H or C₁-C₃ alkyl;
   R¹⁰ is H or halogen;
   m is from 1 to 6;
   and t is from 1 to 6.

Embodiment 69. The RNAi agent of embodiment 68, wherein each R⁸ is H, and m is from 1-3.

Embodiment 70. The RNAi agent of embodiment 69, wherein each R⁹ is H and t is from 1-3.

Embodiment 71. The RNAi agent of embodiment 68, wherein R¹ is methoxypropyl.

Embodiment 72. The RNAi agent of any of embodiments 61-67, wherein R¹ is C₁-C₆ alkyl.

Embodiment 73. The RNAi agent of embodiment 72, wherein R¹ is propyl or isopropyl.

Embodiment 74. The RNAi agent of embodiment 72, wherein R¹ is propyl.

Embodiment 75. The RNAi agent of embodiment 72, wherein R¹ is isobutyl.

Embodiment 76. The RNAi agent of embodiment 72, wherein R¹ is cyclohexyl.

Embodiment 77. The RNAi agent of any of embodiments 61-65, wherein n is 0 and Y is cyclopropyl.

Embodiment 78. The RNAi agent of any of embodiments 61-77, wherein G is selected from OMe, F, or H.

Embodiment 79. The RNAi agent of embodiment 78, wherein G is F.

Embodiment 80. The RNAi agent of embodiment 78, wherein G is OMe.

Embodiment 81. The RNAi agent of embodiment 78, wherein G is H.

Embodiment 82. The RNAi agent of any of embodiments 61-81, having exactly one region having Formula I.

Embodiment 83. The RNAi agent of embodiment 82, wherein the region having Formula I is within the first 8 nucleosides of the antisense RNAi oligonucleotide.

Embodiment 84. The RNAi agent of embodiment 82, wherein the region having Formula I includes the 6^{th} nucleoside from the 5′ end of the antisense RNAi oligonucleotide.

Embodiment 85. The RNAi agent of embodiment 82, wherein the region having Formula I includes the 7^{th} nucleoside from the 5′ end of the antisense RNAi oligonucleotide.

Embodiment 86. An RNAi agent, comprising an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, and wherein the antisense RNAi oligonucleotide contains at least one internucleoside linkage having Formula II:

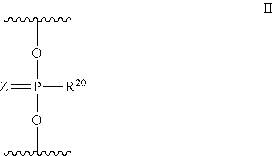

wherein Z is O or S;
R²⁰ is selected from C₁-C₂₀ alkyl, C₁-C₂₀ heteroalkyl, C₃-C₁₀ cycloalkyl, C₆-C₁₀ aryl, 3-10 membered heterocyclyl, and 3-10 membered heteroaryl, and wherein R²⁰ is optionally substituted with C₁-C₂₀alkyl, C₁-C₂₀ heteroalkyl, C₃-C₁₀cycloalkyl, C₆-C₁₀ aryl, 3-10 membered heterocyclyl, 3-10 membered heteroaryl, OR^{D}, NR^{D}R^{D}, SR^{D}, or R^{A}COOR^{D}, wherein R^{A} is C₁-C₆ alkyl and each R^{D} is independently H or C₁-C₆ alkyl.

Embodiment 87. The RNAi agent of Embodiment 86, wherein R²⁰ is R²¹; wherein R²¹ is —[C(R⁸)(R⁹)]ₘ-(Q)ₙ[C(R⁸)(R⁹)]ₜ—R¹⁰ wherein;
Q is O, NR^{D}, or S;
each R⁸ is independently selected from H and C₁-C₃ alkyl;
each R⁹ is independently selected from H and C₁-C₃ alkyl;
R¹⁰ is H, halogen, OR^{D}, NR^{D}R^{D}, SR^{D}, or COOR^{D}, wherein each R^{D} is independently H or C₁-C₃ alkyl;
n is 0 or 1;
m is from 1 to 6; and
and t is from 0 to 6.

Embodiment 88. The RNAi agent of Embodiment 87, wherein Q is O.

Embodiment 89. The RNAi agent of any of embodiments 86-88, wherein Z is O.

Embodiment 90. The RNAi agent of any of embodiments 87-89, wherein n is 1.

Embodiment 91. The RNAi agent of any of embodiments 87-90, wherein R⁸, R⁹, and R¹⁰ are each H.

Embodiment 92. The RNAi agent of any of embodiments 87-91, wherein m is 3.

Embodiment 93. The RNAi agent of any of embodiments 87-92, wherein t is 1.

Embodiment 94. The RNAi agent of any of embodiments 87-93, wherein n is 0.

Embodiment 95. The RNAi agent of embodiment 94, wherein R⁸, R⁹, and R¹⁰ are each H.

Embodiment 96. The RNAi agent of embodiment 94 or 95, wherein m+t equals 3.

Embodiment 97. The RNAi agent of embodiment 86 or 89, wherein R²⁰ is isobutyl.

Embodiment 98. The RNAi agent of embodiment 86 or 89, wherein R²⁰ is propyl.

Embodiment 99. The RNAi agent of embodiment 95, wherein m is 1, t is 0, R⁸ is H and R¹⁰ is COOR^{D}.

Embodiment 100. The RNAi agent of embodiment 99, wherein R^{D} is H.

Embodiment 101. The RNAi agent of embodiment 86 or 89, wherein R²⁰ is methoxypropyl.

Embodiment 102. The RNAi agent of embodiment 86 or 89, wherein R²⁰ is C₃-C₁₀ cycloalkyl.

Embodiment 103. The RNAi agent of embodiment 86 or 89, wherein R²⁰ is cyclohexyl.

Embodiment 104. The RNAi agent of embodiment 86 or 89, wherein R²⁰ is not methyl.

Embodiment 105. The RNAi agent of any of embodiments 86-104, wherein the internucleoside linkage having Formula II is within the seed region of the antisense RNAi oligonucleotide.

Embodiment 106. The RNAi agent of any of embodiments 86-105, wherein the internucleoside linkage having Formula II is between nucleosides 5 to 6 or nucleosides 6 to 7 of the antisense RNAi oligonucleotide, counting from the 5′-end.

Embodiment 107. The RNAi agent of embodiment 106, wherein the internucleoside linkage having Formula II is between nucleosides 6 and 7 of the antisense RNAi oligonucleotide, counting from the 5′ end.

Embodiment 108. The RNAi agent of any of embodiments 86-107, wherein each internucleoside linkage of the antisense RNAi oligonucleotide that does not have Formula II is selected from a phosphorothioate internucleoside linkage and a phosphodiester internucleoside linkage.

Embodiment 109. The RNAi agent of any of embodiments 86-107, wherein each internucleoside linkage of the antisense RNAi oligonucleotide that does not have Formula II is selected from a phosphorothioate internucleoside linkage and a phosphodiester internucleoside linkage, and a mesyl phosphoramidate internucleoside linkage.

Embodiment 110. The RNAi agent of any of embodiments 86-107, wherein the antisense RNAi oligonucleotide consists of 23 linked nucleosides and has an internucleoside linkage motif selected from ssooqoooooooooooooooss or ssoooqooooooooooooooss, wherein each “s” represents a phosphorothioate internucleoside linkage, each “o” represents a phosphodiester internucleoside linkage, and each “q” represents an internucleoside linkage of Formula II.

Embodiment 111. The RNAi agent of embodiment 110, wherein each “q” represents a methoxypropyl internucleoside linkage.

Embodiment 112. The RNAi agent of any of embodiments 61-111, wherein the antisense RNAi oligonucleotide consists of 21 linked nucleosides.

Embodiment 113. The RNAi agent of any of embodiments 61-111, wherein the antisense RNAi oligonucleotide consists of 23 linked nucleosides.

Embodiment 114. The RNAi agent of any of embodiments 61-113, wherein the sense RNAi oligonucleotide consists of 19 linked nucleosides.

Embodiment 115. The RNAi agent of any of embodiments 61-113, wherein the sense RNAi oligonucleotide consists of 21 linked nucleosides.

Embodiment 116. The RNAi agent of any of embodiments 61-115, wherein the antisense RNAi oligonucleotide comprises a 5′-stabilized phosphate moiety.

Embodiment 117. The RNAi agent of embodiment 116, wherein the stabilized phosphate moiety is a 5′-vinyl phosphonate, a 5′-methylene phosphonate, or a 5′-cyclopropyl phosphonate.

Embodiment 118. The RNAi agent of any of embodiments 61-117, wherein the antisense RNAi oligonucleotide does not comprise a GNA or a UNA nucleoside.

Embodiment 119. The RNAi agent of any of embodiments 61-118, wherein each nucleoside of the antisense RNAi oligonucleotide comprises a sugar moiety selected from 2′-fluororibosyl, 2′-O-methyl, 2′-deoxyribosyl, 2′-O-methoxyethyl, or an FHNA sugar surrogate.

Embodiment 120. The RNAi agent of any of embodiments 61-119, wherein the 5′-most nucleoside of the antisense RNAi oligonucleotide comprises a 5′-vinylphosphonate-2′-O-methoxyethyl-3-D-ribosyl sugar moiety.

Embodiment 121. The RNAi agent of any of embodiments 61-120, wherein the nucleoside immediately to the 5′ of the region of Formula I or internucleoside linkage having Formula II comprises a 2′-fluororibosyl or 2′-β-D-deoxyribosyl sugar moiety.

Embodiment 122. The RNAi agent of any of embodiments 61-85, wherein each internucleoside linkage of the antisense RNAi oligonucleotide other than the region having Formula I is selected from a phosphorothioate internucleoside linkage and a phosphodiester internucleoside linkage.

Embodiment 123. The RNAi agent of any of embodiments 61-85, wherein each internucleoside linkage of the antisense RNAi oligonucleotide other than the region having Formula I is selected from a phosphorothioate internucleoside linkage, a phosphodiester internucleoside linkage, and a mesyl phosphoramidate internucleoside linkage.

Embodiment 124. A population of RNAi agents of any of embodiments 61-123, wherein the population is chirally enriched for oligonucleotides having a particular stereochemical configuration at one or more internucleoside linkages.

Embodiment 125. The population of embodiment 124, wherein the stereochemically enriched internucleoside linkage is in the region of Formula I or the internucleoside linkage of Formula II.

Embodiment 126. The population of embodiment 125, wherein the internucleoside linkage in the region of Formula I or the internucleoside linkage of Formula II is in the R_{P} configuration.

Embodiment 127. The population of embodiment 125, wherein the internucleoside linkage in the region of Formula I or the internucleoside linkage of Formula II is in the S_{P} configuration.

Embodiment 128. A population of RNAi agents of any of embodiments 61-123, wherein the stereochemical configuration at the internucleoside linkage in the region of Formula I or the internucleoside linkage of Formula II is stereorandom.

Embodiment 129. A pharmaceutical composition comprising the RNAi agent or population of any of embodiments 61-128 and a pharmaceutically acceptable carrier or diluent.

Embodiment 130. A method comprising contacting a cell with the RNAi agent or population of any of embodiments 61-128 or pharmaceutical composition of embodiment 129.

Embodiment 131. A method of modulating the amount or activity of a target nucleic acid in a cell, comprising contacting the cell with the RNAi agent of any of embodiments 61-128 or pharmaceutical composition of embodiment 129 and thereby modulating the amount or activity of the target nucleic acid.

Embodiment 132. A method of modulating the amount or activity of a target nucleic acid in a cell, comprising contacting the cell with the RNAi agent of any of embodiments 61-128 or pharmaceutical composition of embodiment 129.

Embodiment 133. The method of embodiments 131 or 132, wherein the amount or activity of a target nucleic acid is reduced.

Embodiment 134. Use of the RNAi agent, population, or composition of any of embodiments 61-129 for treatment of a disease or condition.

Embodiment 135. Use of the RNAi agent, population, or composition of any of embodiments 61-129 for a preparation of a medicament for treatment of a disease or condition.

### CERTAIN COMPOUNDS

In certain embodiments, compounds described herein are oligomeric compounds (including oligomeric compounds that are antisense agents or portions thereof) comprising or consisting of oligonucleotides consisting of linked nucleosides and having at least one region having Formula I:

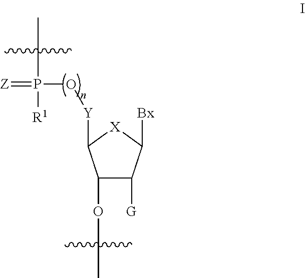

wherein
X is O or O—CH₂;
Z is O or S,
R¹ is selected from C₁-C₂₀ alkyl, C₁-C₂₀ heteroalkyl, C₃-C₁₀ cycloalkyl, C₆-C₁₀ aryl, 3-10 membered heterocyclyl, and 3-10 membered heteroaryl, and wherein R¹ is optionally substituted with C₁-C₂₀ alkyl, C₁-C₂₀ heteroalkyl, C₃-C₁₀ cycloalkyl, C₆-C₁₀ aryl, 3-10 membered heterocyclyl, 3-10 membered heteroaryl, OR^{D}, NR^{D}R^{D}, SR^{D}, or R^{A}COOR^{D}, wherein R^{A} is C₁-C₆ alkyl and each R^{D} is independently H or C₁-C₆ alkyl;
n is 0 or 1;
Y is C₁-C₆ alkyl, C₃-C₁₀ cycloalkyl, or 3-10 membered heterocyclyl; and
each G is independently H, halogen, OH, or O[C(R⁴)(R⁵)]ₚ—[(C═O)_{q}—R⁶]ⱼ—R⁷; wherein
each R⁴ and R⁵ is, independently, H, halogen, C₁-C₆ alkyl or substituted C₁-C₆ alkyl; each R⁶ is O, S or N(E¹);
R⁷ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, or N(E²)(E³);
E¹, E² and E³ are each, independently, H, C₁-C₆ alkyl, or substituted C₁-C₆ alkyl;
p is from 1 to 6;
g is 0 or 1;
j is 0 or 1;
each substituted group comprises one or more optionally protected substituent groups independently selected from halogen, OJ¹, N(J¹)(J²), =NJ¹, SJ¹, N₃, CN, OC(=Q)J¹, OC(=Q)N(J¹)(J²), and C(=Q)N(J¹)(J²);
Q is O, S or NJ³;
J¹, J² and J³ are each, independently, H or C₁-C₆ alkyl;
Bx is a heterocyclic base moiety.

In certain embodiments, R¹ is —[C(R⁸)(R⁹)]ₘ-(Q)ₙ[C(R⁸)(R⁹)]ₜ—R¹⁰ wherein
Q is O, NR^{D}, or S;
each R⁸ is independently selected from H and C₁-C₃ alkyl;
each R⁹ is independently selected from H and C₁-C₃ alkyl;
R¹⁰ is H, halogen, OR^{D}, NR^{D}R^{D}, SR^{D} or COOR^{D}, wherein R^{D} is H or C₁-C₃ alkyl;
n is 0 or 1;
m is from 1 to 6;
and t is from 0 to 6.

In certain embodiments, Q is O.

In certain embodiments, R¹ is bound at a carbon atom of R¹.

In certain embodiments, R¹ is not methyl.

In certain embodiments, R¹ is selected from C₁-C₂₀ alkyl, C₁-C₂₀ heteroalkyl, C₃-C₁₀ cycloalkyl, C₆-C₁₀ aryl, 3-10 membered heterocyclyl, and 3-10 membered heteroaryl, and wherein R¹ is optionally substituted with C₁-C₂₀ alkyl, C₁-C₂₀ heteroalkyl, C₃-C₁₀ cycloalkyl, C₆-C₁₀ aryl, 3-10 membered heterocyclyl, 3-10 membered heteroaryl, or R^{A}COOR^{D}, wherein R^{A} is C₁-C₆ alkyl and each R^{D} is independently H or C₁-C₆ alkyl;

In certain embodiments, compounds described herein are oligomeric compounds (including oligomeric compounds that are antisense agents or portions thereof) comprising or consisting of oligonucleotides consisting of linked nucleosides and having at least internucleoside linkage having Formula II:

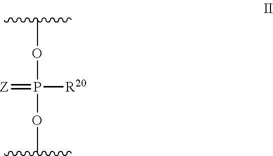

wherein Z is O or S;
R²⁰ is selected from C₁-C₂₀ alkyl, C₁-C₂₀ heteroalkyl, C₃-C₁₀ cycloalkyl, C₆-C₁₀ aryl, 3-10 membered heterocyclyl, and 3-10 membered heteroaryl, and wherein R²⁰ is optionally substituted with C₁-C₂₀ alkyl, C₁-C₂₀ heteroalkyl, C₃-C₁₀ cycloalkyl, C₆-C₁₀ aryl, 3-10 membered heterocyclyl, 3-10 membered heteroaryl, OR^{D}, NR^{D}R^{D}, SR^{D}, or R^{A}COOR^{D}, wherein R^{A} is C₁-C₆ alkyl and R^{D} is independently H or C₁-C₆ alkyl.

In certain embodiments, R²⁰ is R²¹; wherein R²¹ is —[C(R⁸)(R⁹)]ₘ-(Q)ₙ[C(R⁸)(R⁹)]ₜ—R¹⁰ wherein;
Q is O, NR^{D}, or S;
each R⁸ is independently selected from H and C₁-C₃ alkyl;
each R⁹ is independently selected from H and C₁-C₃ alkyl;
R¹⁰ is H, halogen, OR^{D}, NR^{D}R^{D}, SR^{D}, or COOR^{D}, wherein R^{D} is H or C₁-C₃ alkyl;
n is 0 or 1;
m is from 1 to 6;
and t is from 0 to 6.

In certain embodiments, Q is O.

In certain embodiments, R²⁰ is C₃-C₁₀ cycloalkyl.

In certain embodiments, R²⁰ is not methyl.

In certain embodiments, R¹⁰ is H, halogen, or COOR^{D}, wherein R^{D} is H or C₁-C₃ alkyl.

In certain embodiments, R¹⁰ is H.

In certain embodiments, an antisense RNAi oligonucleotide may comprise at least one region having any of Formula Ia-Ig

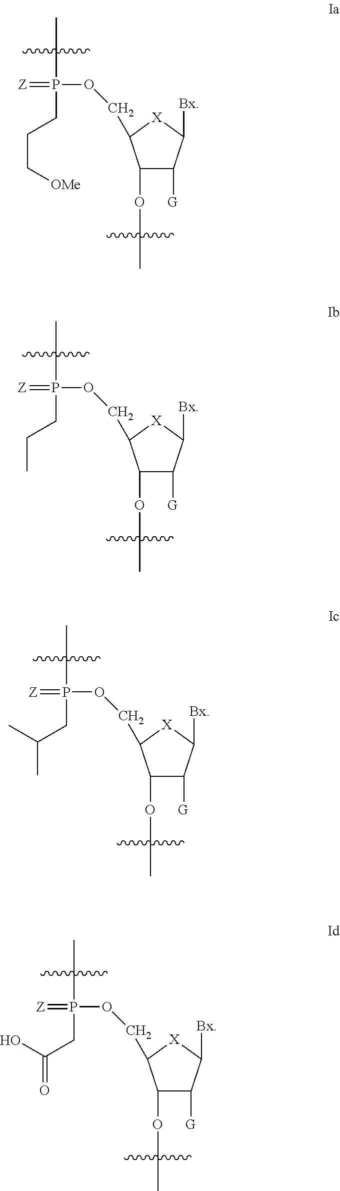

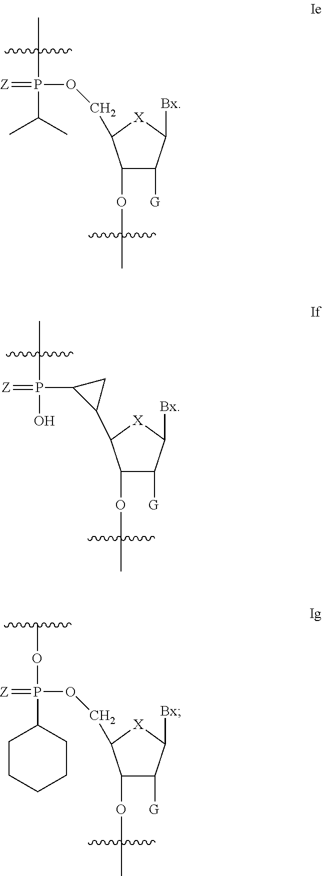

wherein:
X is O or O—CH₂;
Z is O or S;
each G is independently H, halogen, OH, or O[C(R⁴)(R⁵)]ₚ—[(C═O)_{q}—R⁶]—R⁷; wherein
   each R⁴ and R⁵ is, independently, H, halogen, C₁-C₆ alkyl or substituted C₁-C₆ alkyl;
   each R⁶ is O, S or N(E¹);
   R⁷ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, or N(E²)(E³);
   E¹, E² and E³ are each, independently, H, C₁-C₆ alkyl, or substituted C₁-C₆ alkyl;
   p is from 1 to 6;
   q is 0 or 1;
   j is 0 or 1;
   each substituted group comprises one or more optionally protected substituent groups independently selected from halogen, OJ¹, N(J¹)(J²), =NJ¹, SJ¹, N₃, CN, OC(=Q)J¹, OC(=Q)-N(J¹)(J²), and C(=Q)N(J¹)(J²);
      Q is O, S or NJ³;
      J¹, J² and J³ are each, independently, H or C₁-C₆ alkyl;
Bx is a heterocyclic base moiety.

In certain embodiments, an antisense RNAi oligonucleotide may comprise at least one internucleoside linkage having Formula IIa, IIb, IIc, IId, IIe, or IIf

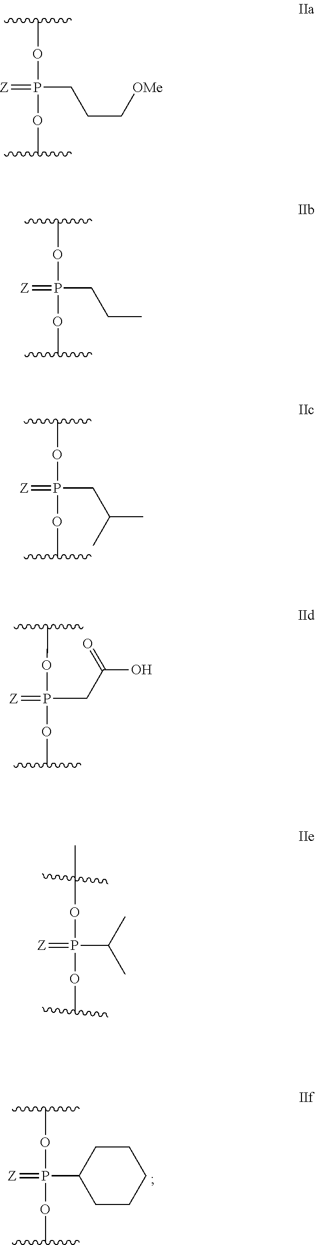

wherein Z is O or S.

In certain embodiments, the 3′-adjacent nucleoside to the region of Formula I comprises a 2′-OMe, 2′-F, or 2′-deoxy sugar moiety. In certain embodiments, the 3′-adjacent nucleoside to the region of Formula I comprises a 2′-OMe or 2′-F sugar moiety.

In certain embodiments, the 3′-adjacent nucleoside to the internucleoside linkage of Formula II comprises a 2′-OMe, 2′-F, or 2′-deoxy sugar moiety. In certain embodiments, the 3′-adjacent nucleoside to the internucleoside linkage of Formula II comprises a 2′-OMe or 2′-F sugar moiety.

### I. Modifications

### A. Modified Nucleosides

Modified nucleosides comprise a stereo-non-standard nucleoside, or a modified sugar moiety, or a modified nucleobase, or any combination thereof.

### 1. Certain Modified Sugar Moieties

In certain embodiments, modified sugar moieties are stereo-non-standard sugar moieties. In certain embodiments, sugar moieties are substituted furanosyl stereo-standard sugar moieties. In certain embodiments, modified sugar moieties are bicyclic or tricyclic furanosyl sugar moieties. In certain embodiments, modified sugar moieties are sugar surrogates. Such sugar surrogates may comprise one or more substitutions corresponding to those of other types of modified sugar moieties.

Certain stereo-non-standard sugar moieties have been previously described in, e.g., Seth et al., WO2020/072991, Seth et al., WO2021/030763, and Seth et al., WO2019/157531, each of which is incorporated by reference herein in their entirety.

In certain embodiments, modified sugar moieties are substituted stereo-standard furanosyl sugar moieties comprising one or more acyclic substituent, including but not limited to substituents at the 2′, 3′, 4′, and/or 5′ positions. In certain embodiments, the furanosyl sugar moiety is a ribosyl sugar moiety. In certain embodiments one or more acyclic substituent of substituted stereo-standard sugar moieties is branched. Examples of 2′-substituent groups suitable for substituted stereo-standard sugar moieties include but are not limited to: 2′-F, 2′-OCH₃(“2′-OMe” or “2′-O-methyl”), and 2′-O(CH₂)₂OCH₃ (“2′-MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(=O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 3′-substituent groups include 3′-methyl (see Frier, et al., The ups and downs of nucleic acid duplex stability: structure-stability studies on chemically-modified DNA:RNA duplexes. Nucleic Acids Res., 25, 4429-4443, 1997.) Examples of 4′-substituent groups suitable for substituted stereo-standard sugar moieties include but are not limited to alkoxy (e.g., methoxy), alkyl, and those described in Manoharan et al., WO 2015/106128. Examples of 5′-substituent groups suitable for substituted stereo-standard sugar moieties include but are not limited to: 5′-methyl (R or S), 5′-allyl, 5′-ethyl, 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836. 2′,4′-difluoro modified sugar moieties have been described in Martinez-Montero, et al., Rigid 2′,4′-difluororibonucleosides: synthesis, conformational analysis, and incorporation into nascent RNA by HCV polymerase. J Org. Chem., 2014, 79:5627-5635. Modified sugar moieties comprising a 2′-modification (OMe or F) and a 4′-modification (OMe or F) have also been described in Malek-Adamian, et al., J Org. Chem, 2018, 83: 9839-9849.

In certain embodiments, a 2′-substituted stereo-standard nucleoside comprises a sugar moiety comprising a non-bridging 2′-substituent group selected from: F, NH₂, N₃, OCF₃, OCH₃, SCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(=O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted stereo-standard nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(=O)—N(H)CH₃ (“NMA”).

In certain embodiments, a 2′-substituted stereo-standard nucleoside comprises a sugar moiety comprising a 2′-substituent group selected from: F, OCH₃, and OCH₂CH₂OCH₃.

In certain embodiments, the 4′ O of 2′-deoxyribose can be substituted with a S to generate 4′-thio DNA (see Takahashi, et al., Nucleic Acids Research 2009, 37: 1353-1362). This modification can be combined with other modifications detailed herein. In certain such embodiments, the sugar moiety is further modified at the 2′ position. In certain embodiments the sugar moiety comprises a 2′-fluoro. A thymidine with this sugar moiety has been described in Watts, et al., J. Org. Chem. 2006, 71(3): 921-925 (4′-S-fluoro5-methylarauridine or FAMU).

a. Bicyclic Nucleosides

Certain nucleosides comprise modified sugar moieties that comprise a bridging sugar substituent that forms a second ring resulting in a bicyclic sugar moiety. In certain such embodiments, the bicyclic sugar moiety comprises a 4′ to 2′ bridge between the 4′ and the 2′ furanose ring atoms. In certain such embodiments, the furanose ring is a ribose ring. Examples of sugar moieties comprising such 4′ to 2′ bridging sugar substituents include but are not limited to bicyclic sugars comprising: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O- 2′ (referred to as “constrained ethyl” or “cEt” when in the S configuration), 4′-CH₂—O—CH₂-2′, 4′-CH₂—N(R)-2′, 4-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672), 4′-C(═O)—N(CH₃)₂-2′, 4′-C(═O)—N(R)₂-2′, 4′-C(═S)—N(R)₂-2′ and analogs thereof (see. e.g., Obika et al., WO2011052436A1, Yusuke, WO2017018360A1).

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2017, 129, 8362-8379; Elayadi et al.,; Christiansen, et al., J. Am. Chem. Soc. 1998, 120, 5458-5463; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

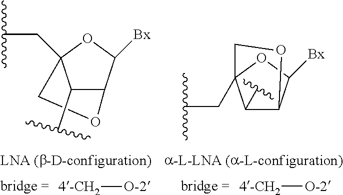

α-L-methyleneoxy (4′-CH₂—O-2′) or α-L-LNA bicyclic nucleosides have been incorporated into antisense oligonucleotides that showed antisense activity (Frieden et alt, Nucleic Acids Research, 2003, 21, 6365-6372). Herein, general descriptions of bicyclic nucleosides include both isomeric configurations. When the positions of specific bicyclic nucleosides (e.g., LNA) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

The term “substituted” following a position of the furanosyl ring, such as “2′-substituted” or “2′-4′-substituted”, indicates that is the only position(s) having a substituent other than those found in unmodified sugar moieties in oligonucleotides.

b. Sugar Surrogates

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), mannitol nucleic acid (“MNA”) (see, e.g., Leumann, CJ. Bioorg. & Med. Chem. 2002, 10, 841-854), fluoro HNA (“FHNA” or “fluoro hexitol nucleic acid”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran. For FHNA, the corresponding sugar surrogate can be referred to as “3′-fluoro-hexitol sugar surrogate” or “FHNA sugar surrogate”; for ANA, the corresponding sugar moiety can be referred to as “altritol nucleic acid sugar surrogate” or “ANA sugar surrogate”, and for HNA, the corresponding sugar surrogate can be referred to as “hexitol nucleic acid sugar surrogate” or “HNA sugar surrogate”.

In certain embodiments, sugar surrogates comprise rings having no heteroatoms. For example, nucleosides comprising bicyclo [3.1.0]-hexane have been described (see, e.g., Marquez, et al., J. Med. Chem. 1996, 39:3739-3749).

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate comprising the following structure:

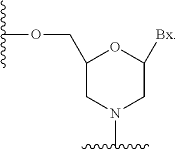

In certain embodiments, morpholinos may be modified, for example by adding or altering various substituent groups from the above morpholino structure. Such sugar surrogates are referred to herein as “modified morpholinos.” In certain embodiments, morpholino residues replace a full nucleotide, including the internucleoside linkage, and have the structures shown below, wherein Bx is a heterocyclic base moiety.

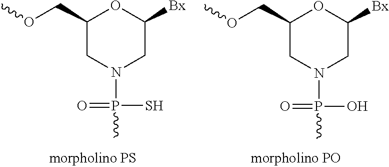

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), glycol nucleic acid (“GNA”, see Schlegel, et al., J. Am. Chem. Soc. 2017, 139:8537-8546) and nucleosides and oligonucleotides described in Manoharan et al., WO2011/133876. Another example of a sugar surrogate containing an acyclic moiety is unlocked nucleic acid (“UNA”), having a structure:

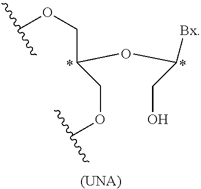

In certain embodiments, acyclic sugar surrogates are selected from:

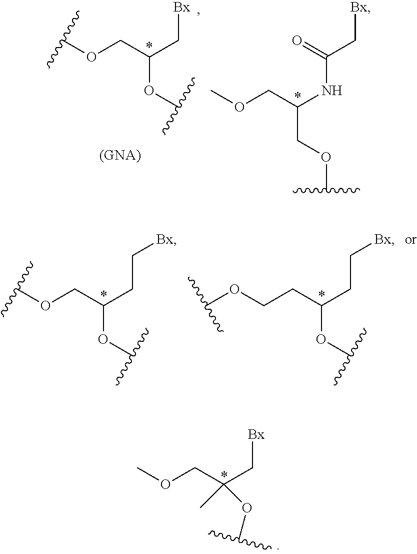

Many other bicyclic and tricyclic sugar and sugar surrogate ring systems are known in the art that can be used in modified nucleosides. Certain such ring systems are described in Hanessian, et al., J. Org. Chem., 2013, 78: 9051-9063 and include bcDNA and tcDNA. Modifications to bcDNA and tcDNA, such as 6′-fluoro, have also been described (Dogovic and Leumann, J. Org. Chem., 2014, 79: 1271-1279).

c. Conjugated Nucleosides and Terminal Groups

In certain embodiments, modified sugar moieties comprise a conjugate group and/or a terminal group. Modified sugar moieties are linked to conjugate groups through a conjugate linker. In certain embodiments, modified furanosyl sugar moieties comprise conjugate groups attached at the 2′, 3′, or 5′ positions. In certain embodiments, the 3′-most sugar moiety of the nucleoside is modified with a conjugate group or a terminal group. In certain embodiments, the 5′-most sugar moiety of the nucleoside is modified with a conjugate group or a terminal group. In certain embodiments, a sugar moiety near the 3′ end of the nucleoside is modified with a conjugate group. In certain embodiments, a sugar moiety near the 5′ end of the nucleoside is modified with a conjugate group.

Examples of terminal groups include but are not limited to conjugate groups, capping groups, phosphate group, protecting groups, modified or unmodified nucleosides, and two or more nucleosides that are independently modified or unmodified.

In certain embodiments, terminal groups at the 5′-terminus comprise a stabilized phosphate group. In certain such embodiments, the phosphorus atom of the stabilized phosphate group is attached to the 5′-terminal nucleoside through a phosphorus-carbon bond. In certain embodiments, the carbon of that phosphorus-carbon bond is in turn bound to the 5′-position of the nucleoside.

In certain embodiments, the oligonucleotide comprises a 5′-stabilized phosphate group having the following formula:

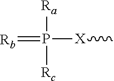

wherein:
Rₐ and R_{c} are each, independently, OH, SH, C₁-C₆ alkyl, substituted C₁-C₆ alkyl, C₁-C₆ alkoxy, substituted C₁-C₆ alkoxy, amino or substituted amino;
R_{b} is O or S;
X is substituted or unsubstituted C; and wherein X is attached to the 5′-terminal nucleoside. In certain embodiments, X is bound to an atom at the 5′-position of the 5′-terminal nucleoside of an antisense RNAi oligonucleotide. In certain such embodiments, the 5′-atom is a carbon and the bond between X and the 5′-carbon of the 5′-terminal nucleoside is a carbon-carbon single bond. In certain embodiments, it is a carbon-carbon double bond. In certain embodiments, it is a carbon-carbon triple bond. In certain embodiments, the 5′-carbon is substituted. In certain embodiments, X is substituted. In certain embodiments, X is unsubstituted.

In certain embodiments, the oligonucleotide comprises a 5′-stabilized phosphate group having the following formula:

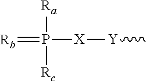

wherein:
Rₐ and R_{c} are each, independently, OH, SH, C₁-C₆ alkyl, substituted C₁-C₆ alkyl, C₁-C₆ alkoxy, substituted C₁-C₆ alkoxy, amino or substituted amino;
R_{b} is O or S;
X is substituted or unsubstituted C;
Y is selected from C, S, and N. In certain embodiments, Y is substituted or unsubstituted C. The bond between X and Y may be a single-, double-, or triple-bond.

Certain 5′-stabilized phosphate groups have been previously described; see, e.g., Prakash et al., WO2011/139699 and Prakash et al., WO2011/139702, hereby incorporated by reference herein in their entirety.

In certain embodiments, the stabilized phosphate group is 5′-vinyl phosphonate, 5′-methylene phosphonate or 5′-cyclopropyl phosphonate.

### 2. Modified Nucleobases

In certain embodiments, a modified oligonucleotide comprises one or more nucleoside comprising an unmodified nucleobase. An “unmodified nucleobase” is unmodified adenine (A), unmodified thymine (T), unmodified cytosine (C), unmodified uracil (U), or unmodified guanine (G). In certain embodiments, a modified oligonucleotide comprises one or more inosine nucleosides (e.g., a nucleoside comprising a hypoxanthine nucleobase).

In certain embodiments, a modified oligonucleotide comprises one or more nucleoside that does not comprise a nucleobase, referred to as an abasic nucleoside.

In certain embodiments, a modified oligonucleotide comprises one or more nucleoside comprising a modified nucleobase. A modified nucleobase is a group of atoms other than unmodified A, T, C, U, or G capable of pairing with at least one other nucleobase. A 5-methylcytosine is an example of a modified nucleobase. A universal base is a modified nucleobase that can pair with any one of the five unmodified nucleobases.

Unless otherwise indicated, modified adenine has structure:

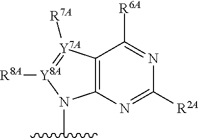

wherein: R^{2A} is H, C₁-C₆ alkyl, substituted C₁-C₆ alkyl, C₁-C₆ thioalkyl, or substituted C₁-C₆ thioalkyl, C₁-C₆ alkyloxy, or substituted C₁-C₆ alkyloxy; R^{6A} is H, N(R^{a})(R^{b}), acetyl, formyl, or O-phenyl; Y^{7A} is N and R^{7A} is absent or is C₁-C₆ alkyl; or Y^{7A} is C and R^{7A} is selected from H, C₁-C₆ alkyl, or N(R^{a})(R^{b}); Y^{8A} is N and R^{8A} is absent, or Y^{8A} is C and R^{8A} is selected from H, a halogen, OH, C₁-C₆ alkyl, or substituted C₁-C₆ alkyl; R^{a} and R^{b} are independently selected from H, C₁-C₆ alkyl, substituted C₁-C₆ alkyl, C₁-C₆ alkenyl, substituted C₁-C₆ alkenyl, acetyl, formyl, or together form a 5-7-membered heterocycle; excluding where Y^{7A} is N; Y^{8A} is C, R^{8A} is H, R^{2A} is H, and R^{6A} is NH₂ (unmodified adenine).

Unless otherwise indicated, modified guanine has structure:

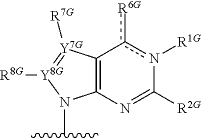

wherein: R^{2G} is N(R^{a})(R^{b}); R^{6G} is oxo and R^{1G} is H, or R^{6G} is selected from O—C₁-C₆ alkyl or S—C₁-C₆ alkyl and R^{1G} is absent; Y^{7G} is N and R^{7A} is absent or is C₁-C₆ alkyl; or Y^{7G} is C and R^{7G} is selected from H, C₁-C₆ alkyl, or N(R^{a})(R^{b}); Y^{8G} is N and R^{8G} is absent, or Y^{8G} is C and R^{8G} is selected from H, a halogen, OH, C₁-C₆ alkyl, or substituted C₁-C₆ alkyl; R^{a} and R^{b} are independently selected from H, C₁-C₆ alkyl, substituted C₁-C₆ alkyl, C₁-C₆ alkenyl, substituted C₁-C₆ alkenyl, acetyl, formyl, or together form a 5-7-membered heterocycle; excluding where Y^{7G} is N; Y^{8G} is C, R^{8G} is H, R^{2G} is NH₂, and R^{6G} is =O (unmodified guanosine).

Unless otherwise indicated, modified thymine or modified uracil has structure:

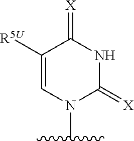

wherein: X is selected from O or S and R^{5U} is selected from H, OH, halogen, O—C₁-C₂ alkyl, O—C₁-C₁₂ substituted alkyl, C₁-C₁₂ alkyl, substituted C₁-C₁₂ alkyl, C₁-C₁₂ alkenyl, substituted C₁-C₁₂ alkenyl, C₁-C₁₂ alkynyl, substituted C₁-C₁₂ alkynyl; wherein if each X is O, R^{5U} is not H or CH₃ (unmodified uracil and unmodified thymine, respectively).

Unless otherwise indicated, modified cytosine has structure:

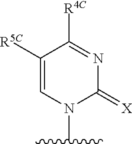

wherein: X is selected from O or S, R^{4C} is N(R^{a})(R^{b}); R^{5C} is selected from H, OH, halogen, O—C₁-C₁₂ alkyl, O—C₁-C₁₂ substituted alkyl, C₁-C₁₂ alkyl, substituted C₁-C₁₂ alkyl, C₁-C₁₂ alkenyl, substituted C₁-C₁₂ alkenyl; R^{a} and R^{b} are independently selected from H, C₁-C₆ alkyl, substituted C₁-C₆ alkyl, C₁-C₆ alkenyl, substituted C₁-C₆ alkenyl, C₁-C₁₂ alkynyl, substituted C₁-C₁₂ alkynyl; acetyl, formyl, or together form a 5-7-membered heterocycle; excluding where X is O, R^{4C} is NH₂ and R^{5C} is H (unmodified cytosine).

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), N1-methylpseudouracil, 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie. International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443. In certain embodiments, modified nucleosides comprise double-headed nucleosides having two nucleobases. Such compounds are described in detail in Sorinas et al., J. Org. Chem, 2014 79: 8020-8030.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, each nucleobase of a modified oligonucleotide is selected from unmodified A, unmodified G, unmodified C, unmodified T, unmodified U, and 5-methyl C. In certain embodiments, there are no modified nucleobases in a modified oligonucleotide and each nucleobase of a modified oligonucleotide is selected from unmodified A, unmodified G, unmodified C, unmodified T, and unmodified U.

In certain embodiments, compounds comprise or consist of a modified oligonucleotide complementary to a target nucleic acid comprising one or more modified nucleobases. In certain embodiments, the modified nucleobase is 5-methylcytosine. In certain embodiments, each cytosine is a 5-methylcytosine.

### B. Modified Internucleoside Linkages

In certain embodiments, antisense agents, oligomeric compounds, and modified oligonucleotides comprise or consist of a modified oligonucleotide complementary to a target nucleic acid comprising one or more modified internucleoside linkages. In certain embodiments, the modified internucleoside linkages are phosphorothioate linkages. In certain embodiments, each internucleoside linkage of an antisense compound is a phosphorothioate internucleoside linkage.

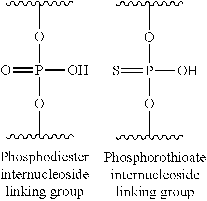

In certain embodiments, nucleosides of modified oligonucleotides may be linked together using any internucleoside linkage. The two main classes of internucleoside linkages are defined by the presence or absence of a phosphorus atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, phosphorothioate, and phosphorodithioate (“HS-P=S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Neutral internucleoside linkages include, without limitation, phosphotriesters, phosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′- O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

a. Chiral Internucleoside Linkages

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. All phosphorothioate linkages described herein are stereorandom unless otherwise specified. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

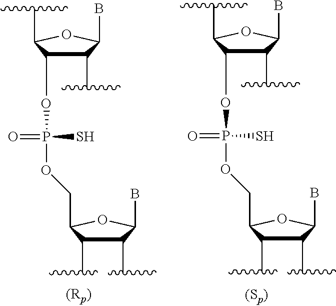

Unless otherwise indicated, chiral internucleoside linkages of modified oligonucleotides described herein can be stereorandom or in a particular stereochemical configuration.
b. Alternatives to 5′ to 3′ Internucleoside Linkages

In certain embodiments, nucleic acids can be linked 2′ to 5′ rather than the standard 3′ to 5′ linkage. Such a linkage is illustrated below.

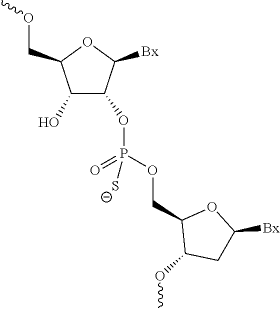

In certain embodiments, nucleosides can be linked by 2′, 3′-phosphodiester bonds. In certain such embodiments, the nucleosides are threofuranosyl nucleosides (TNA; see Bala, et al., J Org. Chem. 2017, 82:5910-5916). A TNA linkage is shown below.

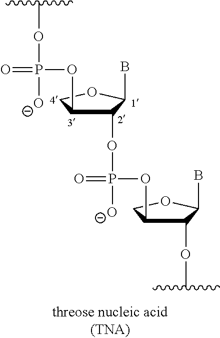

Additional modified linkages include α,β-D-CNA type linkages and related conformationally-constrained linkages, shown below. Synthesis of such molecules has been described previously (see Dupouy et al., Angew. Chem Int. Ed. Engl. 2014, 45: 3623-3627: Borsting, et al. Tetrahedron. 2004, 60:10955-10966; Ostergaard, et al., ACS Chem. Biol. 2014, 9: 1975-1979; Dupouy, et al., Eur. J. Org. Chem., 2008, 1285-1294; Martinez, et al., PLoS One. 2011. 6:e25510; Dupouv, et al., Eur J. Org. Chem., 2007, 5256-5264; Boissonnet, et al., New J. Chem., 2011, 35: 1528-1533.)

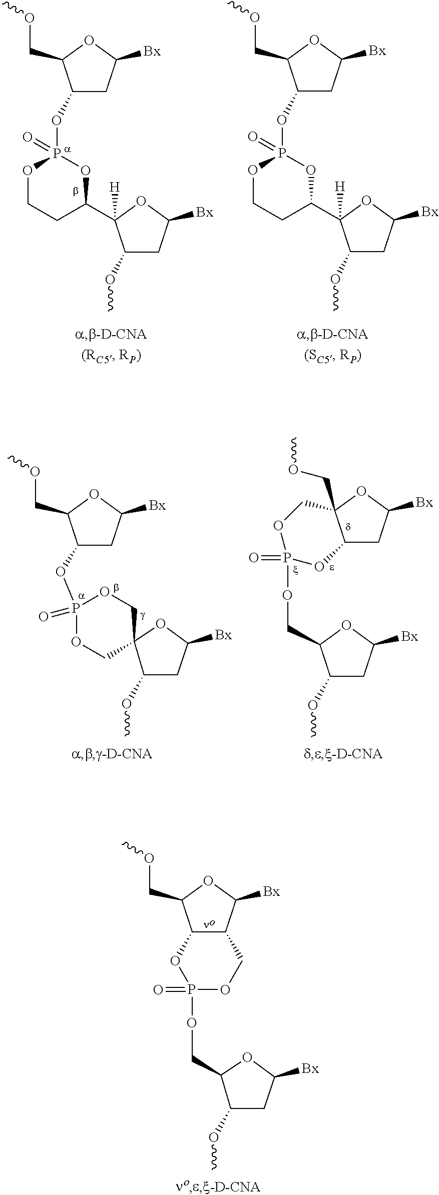

c. Linkages Having Conjugate Groups

In certain embodiments, an internucleoside linking group may comprise a conjugate group. In certain embodiments, an sulfonyl phosphoramidate internucleoside linking group comprises a conjugate group. In certain embodiments, the conjugate group of a modified oligonucleotide may be attached to the remainder of the modified oligonucleotide through a sulfonyl phosphoramidate internucleoside linking group:

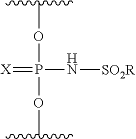

wherein R comprises a conjugate group. In certain embodiments, the conjugate group comprises a cell-targeting moiety. In certain embodiments, the conjugate group comprises a carbohydrate or carbohydrate cluster. In certain embodiments, the conjugate group comprises GalNAc. In certain embodiments, the conjugate group comprises a lipid. In certain embodiments, the conjugate group comprises C₁₀-C₂₀ alkyl. In certain embodiments, the conjugate group comprises C₁₆ alkyl.

In certain embodiments, the internucleoside linking group comprising a conjugate group has Formula III:

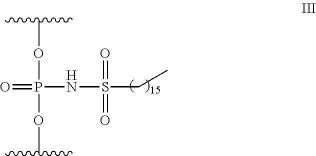

### II. Certain Motifs

In certain embodiments, antisense agents, oligomeric compounds, and modified oligonucleotides described herein comprise or consist of oligonucleotides. Modified oligonucleotides can be described by their motif, e.g. a pattern of unmodified and/or modified sugar moieties, nucleobases, and/or internucleoside linkages. In certain embodiments, modified oligonucleotides comprise one or more stereo-non-standard nucleosides. In certain embodiments, modified oligonucleotides comprise one or more stereo-standard nucleosides. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns or motifs of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### A. Certain Sugar Motifs

In certain embodiments, antisense agents, oligomeric compounds, and modified oligonucleotides described herein comprise or consist of oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include without limitation any of the sugar modifications discussed herein.

In certain embodiments, each nucleoside of a modified oligonucleotide, or portion thereof, comprises a 2′-substituted sugar moiety, a bicyclic sugar moiety, a sugar surrogate, or a 2′-deoxyribosyl sugar moiety. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, PNA, THP, and FHNA.

In certain embodiments, modified oligonucleotides comprise at least 12, at least 13, at least 14, at least 15, at least 16, at least 17, at least 18, at least 19, or at least 20 nucleosides comprising a modified sugar moiety. In certain embodiments, the modified sugar moiety is selected independently from a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and FHNA.

Provided herein is an antisense RNAi oligonucleotide comprising a region of Formula I, or having having at least internucleoside linkage of Formula II, in the seed region thereof. Also provided is an RNAi agent comprising an antisense RNAi oligonucleotide comprising a region of Formula I. or having having at least internucleoside linkage of Formula II, in the seed region thereof. In certain embodiments, the region of Formula I has a structure of one of Formula Ia-Ig. An RNAi agent of the disclosure may be an oligomeric duplex comprising an antisense RNAi oligomeric compound comprising an antisense RNAi oligonucleotide and a sense RNAi oligomeric compound comprising a sense RNAi oligonucleotide. Generally, the remainder of the internucleoside linkages in the RNAi agent are chosen from phosphodiester and phosphorothioate, however, an RNAi agent provided herein may comprise other, optionally one or more, internucleoside linkages, where the other internucleoside linkages may be as described herein or as known in the art. Both the antisense RNAi oligonucleotide and the sense RNAi oligonucleotide are characterized by respective motifs of sugar moieties which can be conceptually separated from the nucleobase sequences thereof. The motif or sequence of sugar moieties is known to affect loading into protein complexes which are relevant to knockdown of a target mRNA or pre-mRNA. See, e.g., Hu, B., et al Therapeutic siRNA: state of the art. Sig Transduct Target Ther 5, 101 (2020).

In certain embodiments, at least one nucleoside of a modified oligonucleotide comprises a 2′-OMe sugar moiety. In certain embodiments, at least 2, at least 5, at least 8, at least 10, at least 12, at least 13, at least 14, at least 15, at least 16, at least 17, at least 18, at least 19, at least 20, or at least 21 nucleosides comprise a 2′-OMe sugar moiety. In certain embodiments, a modified oligonucleotide comprises one, two, or three blocks of at least 4 contiguous 2′-OMe nucleosides. In certain embodiments, an antisense RNAi oligonucleotide comprises 2′-OMe nucleosides at nucleosides 3-5, counting from the 5′-terminal nucleoside. In certain embodiments, an antisense RNAi oligonucleotide comprises 2′-OMe nucleosides at nucleosides 8-13. In certain embodiments, an antisense RNAi oligonucleotide comprises 2′-OMe nucleosides at nucleosides 15 and 17-19. In certain embodiments, an antisense RNAi oligonucleotide comprises 2′-OMe nucleosides at nucleosides 3-13. In certain embodiments, each 2′-OMe nucleoside of a modified oligonucleotide comprises a ribo-2′-OMe sugar moiety. In certain such embodiments the remainder of the nucleosides in the modified oligonucleotide are selected from 2′-deoxynucleosides, 2′-F nucleosides, 2′-MOE nucleosides, and 2′-OMe nucleosides. In any embodiment of this paragraph, a 2′-OMe nucleoside may be a constituent of a region of Formula I.

In certain embodiments, at least one nucleoside of a modified oligonucleotide comprises a 2′-F sugar moiety (i.e., a 2′-F modified nucleoside). In certain embodiments, a modified oligonucleotide comprises exactly 1, 2, 3, 4, or 5 nucleosides comprising a 2′-F sugar moiety. In certain embodiments, a modified oligonucleotide comprises a block of 2, 3, or 2-4 contiguous 2′-F nucleosides. In certain embodiments, an antisense RNAi oligonucleotide comprises a 2′-F nucleoside at one, two, three, or four of nucleosides 2, 6, 14, and/or 16, counting from the 5′-terminal nucleoside. In certain embodiments, an antisense RNAi oligonucleotide comprises a 2′-F nucleoside at one, two, or three of nucleosides 2, 14, and/or 16. In certain embodiments, an antisense RNAi oligonucleotide comprises a 2′-F nucleoside at one or two of nucleosides 2 and/or 14. In certain such embodiments the remainder of the nucleosides in the modified oligonucleotide are selected from 2′-deoxynucleosides, 2′-MOE nucleosides, and 2′-OMe nucleosides. In certain such embodiments the remainder of the nucleosides in the modified oligonucleotide are ribo-2′-OMe modified nucleosides. In certain embodiments, each 2′-F nucleoside of a modified oligonucleotide comprises a ribo-2′-F sugar moiety. In certain embodiments, all but one 2′-F nucleoside of a modified oligonucleotide comprises a ribo-2′-F sugar moiety. In any of the embodiments described herein, a modified oligonucleotide may comprise a nucleoside comprising an FHNA sugar surrogate. In any of the embodiments described herein comprising a 2′-F nucleoside, one, two, three, one or more, or all such 2′-F nucleoside may be replaced with a nucleoside comprising an FHNA sugar surrogate. In any embodiment of this paragraph, a 2′-F nucleoside may be a constituent of a region of Formula I.

In certain embodiments, the modified oligonucleotide comprises 1, 2, or 3 2′-deoxy sugar moieties. In certain embodiments, each 2′-deoxynucleoside of a modified oligonucleotide comprises a 2′-β-D-deoxyribosyl sugar moiety. In certain embodiments, all but one 2′-deoxynucleoside nucleoside of a modified oligonucleotide comprises a 2′-β-D-deoxyribosyl sugar moiety. In certain embodiments, an antisense RNAi oligonucleotide comprises a 2′-deoxy nucleoside at one or two of nucleosides 5, 6, and 7, counting from the 5′-terminal nucleoside. In certain embodiments, an antisense RNAi oligonucleotide comprises a 2′-deoxy nucleoside at nucleoside 6. In certain embodiments, an antisense RNAi oligonucleotide comprises a 2′-deoxy nucleoside at nucleosides 5 and 7. In certain such embodiments the remainder of the nucleosides in the modified oligonucleotide are selected from 2′-F nucleosides, 2′-MOE nucleosides, and 2′-OMe nucleosides. In any embodiment of this paragraph, a 2′-deoxynucleoside may be a constituent of a region of Formula I.

In certain embodiments, the modified oligonucleotide comprises 1, 2, 3, 4 or 5 2′-MOE sugar moieties. In certain embodiments, each 2′-MOE nucleoside of a modified oligonucleotide comprises a ribo-2′-MOE sugar moiety. In certain embodiments, all but one 2′-MOE nucleoside of a modified oligonucleotide comprises a ribo-2′-MOE sugar moiety. In certain embodiments, an antisense RNAi oligonucleotide comprises a 2′-MOE nucleoside at one, two, three, four, or five of nucleosides 1, 9, 10, 22, and 23, counting from the 5′-terminal nucleoside. In certain embodiments, an antisense RNAi oligonucleotide comprises a 2′-MOE nucleoside at nucleoside 1. In certain embodiments, an antisense RNAi oligonucleotide comprises a 2′-MOE nucleoside at one or two of nucleosides 9 and/or 10. In certain embodiments, an antisense RNAi oligonucleotide comprises a 2′-MOE nucleoside at one or two of nucleosides 22 and/or 23. In certain such embodiments the remainder of the nucleosides in the modified oligonucleotide are selected from 2′-deoxy nucleosides, 2′-F nucleosides, and 2′-OMe nucleosides. In any embodiment of this paragraph, a 2′-MOE nucleoside may be a constituent of a region of Formula I.

Certain RNAi motifs are described in, e.g., Freier, et al., WO2020/160163, incorporated by reference herein in its entirety; as well as, e.g., Rajeev, et al., WO2013/075035; Maier, et al., WO2016/028649; Theile, et al., WO2018/098328; Nair, et al., WO2019/217459; each of which is incorporated by reference herein.

In certain embodiments, a sugar motif of the antisense RNAi oligonucleotide is selected from those described in WO 2022/174053, wherein one nucleoside in the seed region is replaced with a region of Formula I, or alternatively where one internucleoside linkage is replaced with an internucleoside linkage of Formula II as described herein. In certain embodiments, a sugar motif of the sense RNAi oligonucleotide is selected from those described in WO 2022/174053.

In certain embodiments, a modified oligonucleotide is a uniformly modified oligonucleotide in which each modified nucleoside comprises the same 2′-modification. In certain embodiments, every second nucleoside of a uniformly modified nucleotide comprises the same 2′-modification, providing alternating 2′-modifications. In certain such embodiments, the 2′ modifications are 2′-OMe and 2′-F (i.e., alternating 2′-OMe and 2′-F nucleosides).

### B. Certain Nucleobase Motifs

In certain embodiments antisense agents, oligomeric compounds, and modified oligonucleotides described herein comprise or consist of oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, one nucleoside comprising a modified nucleobase is in the central region of a modified oligonucleotide. In certain such embodiments, the sugar moiety of said nucleoside is a 2′-β-D-deoxyribosyl moiety. In certain such embodiments, the modified nucleobase is selected from: 5-methyl cytosine, 2-thiopyrimidine, 2-thiothymine, 6-methyladenine, inosine, pseudouracil, or 5-propynepyrimidine.

### C. Certain Internucleoside Linkage Motifs

In certain embodiments, antisense agents, oligomeric compounds, and modified oligonucleotides described herein comprise or consist of oligonucleotides. In certain embodiments, oligonucleotides comprise modified and/or unmodified internucleoside linkages arranged along the oligonucleotide or region thereof in a defined pattern or motif Such oligonucleotides comprise a region of Formula I or an internucleoside linkage of Formula II as described herein.

In certain embodiments, each phosphorothioate internucleoside linkage is independently selected from a stereorandom phosphorothioate, a (Sp) phosphorothioate, and a (Rp) phosphorothioate. In certain embodiments, the internucleoside linkages within the central region of a modified oligonucleotide are all modified. In certain such embodiments, all of the phosphorothioate linkages are stereorandom. In certain embodiments, all of the phosphorothioate linkages in the 5′-region and 3′-region are (Sp) phosphorothioates, and the central region comprises at least one Sp, Sp, Rp motif. In certain embodiments, populations of modified oligonucleotides are enriched for modified oligonucleotides comprising such internucleoside linkage motifs.

In certain embodiments, a double-stranded antisense agent is a double-stranded RNAi duplex comprising an antisense RNAi oligomeric compound and a sense RNAi oligomeric compound, wherein one or both of the antisense RNAi oligonucleotide and/or sense RNAi oligomeric compound have one or more mesyl phosphoramidate internucleoside linkages. In certain embodiments, the RNAi antisense oligonucleotide comprises at least two, at least three, at least four, at least five, or at least six mesyl phosphoramidate internucleoside linkages. In certain embodiments, the sense RNAi oligonucleotide comprises at least two, at least three, at least four, at least five, or at least six mesyl phosphoramidate internucleoside linkages.

### D. Certain Modified Oligonucleotides

In certain embodiments, antisense agents, oligomeric compounds, and modified oligonucleotides described herein comprise or consist of modified oligonucleotides. In certain embodiments, the above modifications (sugar, nucleobase, internucleoside linkage) are incorporated into a modified oligonucleotide. In certain embodiments, modified oligonucleotides are characterized by their modifications, motifs, and overall lengths. In certain embodiments, such parameters are each independent of one another. Thus, unless otherwise indicated, each internucleoside linkage of a modified oligonucleotide may be modified or unmodified and may or may not follow the modification pattern of the sugar moieties. Likewise, such modified oligonucleotides may comprise one or more modified nucleobase independent of the pattern of the sugar modifications. Furthermore, in certain instances, a modified oligonucleotide is described by an overall length or range and by lengths or length ranges of two or more regions (e.g., a region of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions or segments, A, B, and C, wherein region or segment A consists of 2-6 linked nucleosides having a specified sugar moiety, region or segment B consists of 6-10 linked nucleosides having a specified sugar moiety, and region or segment C consists of 2-6 linked nucleosides having a specified sugar moiety. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of 20 for the overall length of the modified oligonucleotide. Unless otherwise indicated, all modifications are independent of nucleobase sequence except that the modified nucleobase 5-methylcytosine is necessarily a “C” in an oligonucleotide sequence. In certain embodiments, when a DNA nucleoside or DNA-like nucleoside that comprises a T in a DNA sequence is replaced with a RNA-like nucleoside, the nucleobase T is replaced with the nucleobase U. Each of these compounds has an identical target RNA.

In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments oligonucleotides have a nucleobase sequence that is complementary to a second oligonucleotide or an identified reference nucleic acid, such as a target nucleic acid. In certain embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

### III. Certain Conjugated Compounds

In certain embodiments, antisense agents, oligomeric compounds, and modified oligonucleotides described herein comprise or consist of a modified oligonucleotide that optionally comprises a conjugate group. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate moieties or terminal groups are attached at the 3′ and/or 5′-end of oligonucleotides. In certain such embodiments, conjugate moieties (or terminal groups) are attached at the 3′-end of oligonucleotides. In certain embodiments, conjugate moieties are attached near the 3′-end of oligonucleotides. In certain embodiments, conjugate moieties (or terminal groups) are attached at the 5′-end of oligonucleotides. In certain embodiments, conjugate moieties are attached near the 5′-end of oligonucleotides.

In certain embodiments, at least one internucleoside linkage is a sulfonyl phosphoramidate internucleoside linking group:

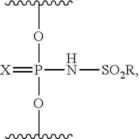

Wherein R comprises a conjugate group. In certain embodiments, R is C₁₆. In certain embodiments, R is a linear or branched C₁₆₋₂₂, e.g., a linear C₁₆ or a branched C₂₂.

### A. Certain Conjugate Groups and Conjugate Moieties

In certain embodiments, modified oligonucleotides comprise one or more conjugate moieties or conjugate groups. In certain embodiments, conjugate groups modify one or more properties of the molecule, including but not limited to pharmacodynamics, pharmacokinetics, stability, binding, absorption, tissue distribution, cellular distribution, cellular uptake, charge and clearance. In certain embodiments, conjugate moieties impart a new property on the molecule, e.g., fluorophores or reporter groups that enable detection of the molecule.

Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta. 1995, 1264, 229-237), an octadecylamine or hexylamino-carbonyl-oxycholesterol moiety (Crooke et al., J. Pharmacol. Exp. Ther., 1996, i, 923-937),_a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

a. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes. In certain embodiments, the conjugate moiety comprises a bicyclic peptide, e.g., as described in WO2023/056388.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

b. Conjugate Linkers

In certain embodiments, conjugate groups comprise a conjugate linker that attaches a conjugate moiety to the remainder of the modified oligonucleotide. In certain embodiments, a conjugate linker is a single chemical bond (i.e. conjugate moiety is attached to the remainder of the modified oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group or conjugate moiety to be cleaved from the remainder of the oligonucleotide. For example, in certain circumstances oligomeric compounds (including oligomeric compounds that are antisense agents or portions thereof) or modified oligonucleotides comprising a particular conjugate moiety are better taken up by a particular cell type, but once the compound has been taken up, it is desirable that the conjugate group be cleaved to release an unconjugated oligonucleotide. Thus, certain conjugate moieties may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is a nucleoside comprising a 2′-deoxyfuranosyl that is attached to either the 3′ or 5′-terminal nucleoside of an oligonucleotide by a phosphodiester internucleoside linkage and covalently attached to the remainder of the conjugate linker or conjugate moiety by a phosphodiester or phosphorothioate linkage. In certain such embodiments, the cleavable moiety is a nucleoside comprising a 2′-β-D-deoxyribosyl sugar moiety. In certain such embodiments, the cleavable moiety is 2′-deoxyadenosine.

c. Certain Cell-Targeting Conjugate Moieties

In certain embodiments, a conjugate group comprises a cell-targeting conjugate moiety. In certain embodiments, a conjugate group has the general formula:

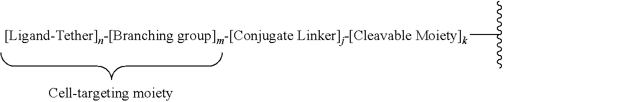

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycolyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, oligomeric compounds (including oligomeric compounds that are antisense agents or portions thereof) or modified oligonucleotides described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

### Compositions and Methods for Formulating Pharmaceutical Compositions

Antisense agents, oligomeric compounds, and modified oligonucleotides described herein may be admixed with pharmaceutically acceptable active or inert substances for the preparation of pharmaceutical compositions. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more oligomeric compounds (including oligomeric compounds that are antisense agents or portions thereof) or a salt thereof. In certain such embodiments, the pharmaceutical composition comprises a suitable pharmaceutically acceptable diluent or carrier. In certain embodiments, a pharmaceutical composition comprises a sterile saline solution and one or more oligomeric compound. In certain embodiments, such pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

An oligomeric compound described herein complementary to a target nucleic acid can be utilized in pharmaceutical compositions by combining the oligomeric compound with a suitable pharmaceutically acceptable diluent or carrier and/or additional components such that the pharmaceutical composition is suitable for injection. In certain embodiments, a pharmaceutically acceptable diluent is phosphate buffered saline. Accordingly, in one embodiment, employed in the methods described herein is a pharmaceutical composition comprising an oligomeric compound complementary to a target nucleic acid and a pharmaceutically acceptable diluent. In certain embodiments, the pharmaceutically acceptable diluent is phosphate buffered saline. In certain embodiments, the oligomeric compound comprises or consists of a modified oligonucleotide provided herein.

Pharmaceutical compositions comprising oligomeric compounds (including oligomeric compounds that are antisense agents or portions thereof) provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the oligomeric compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

### Certain Mechanisms

In certain embodiments, oligomeric compounds (including oligomeric compounds that are antisense agents or portions thereof) described herein comprise or consist of modified oligonucleotides. In certain such embodiments, the oligomeric compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Nucleosides that are sufficiently “DNA-like” to elicit RNase H activity are referred to as DNA mimics herein. Further, in certain embodiments, one or more non-DNA-like nucleoside in in the RNA:DNA duplex is tolerated.

In certain antisense activities, hybridization of an antisense agent, oligomeric compound, or modified oligonucleotide described herein to a target nucleic acid results in modulation of the splicing of a target pre-mRNA. For example, in certain embodiments, hybridization of a compound described herein will increase exclusion of an exon. For example, in certain embodiments, hybridization of a compound described herein will increase inclusion of an exon.

In certain antisense activities, antisense agents described herein or a portion of the antisense agent is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi agents. RNAi agents may be double-stranded (siRNA) or single-stranded (ssRNA).

In certain antisense activities, antisense agents, oligomeric compounds, or modified oligonucleotides described herein result in a CRISPR system cleaving a target DNA. In certain antisense activities, compounds described herein result in a CRISPR system editing a target DNA.

In certain antisense activities, hybridization of an antisense agent, oligomeric compound, or modified oligonucleotide described herein to a target nucleic acid results in disruption of secondary structural elements, such as stem-loops and hairpins. For example, in certain embodiments, hybridization of a compound described herein to a stem-loop that is part of a translation suppression element leads to an increase in protein expression.

In certain antisense activities, hybridization of an antisense agent, oligomeric compound, or modified oligonucleotide described herein to a target nucleic acid leads to no-go decay mediated mRNA degradation.

In certain antisense activities, hybridization of an antisense agent, oligomeric compound, or modified oligonucleotide described herein to a target nucleic acid leads to activation of nonsense-mediated decay mRNA degradation.

In certain embodiments, antisense agents, oligomeric compounds, or modified oligonucleotides described herein are artificial mRNA compounds, the nucleobase sequence of which encodes for a protein.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Certain RNAi Agents

Certain RNAi motifs are described in, e.g., Freier, et al., WO2020/160163, incorporated by reference herein in its entirety; as well as, e.g., Rajeev, et al., WO2013/075035; Maier, et al., WO2016/028649; Theile, et al., WO2018/098328; Nair, et al., WO2019/217459; each of which is incorporated by reference herein.

### Target Nucleic Acids, Target Regions and Nucleotide Sequences

In certain embodiments, antisense agents, oligomeric compounds, or modified oligonucleotides described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain embodiments, a pre-mRNA and corresponding mRNA are both target nucleic acids of a single compound. In certain such embodiments, the target region is entirely within an intron of a target pre-mRNA. In certain embodiments, the target region spans an intron/exon junction. In certain embodiments, the target region is at least 50% within an intron. In certain embodiments, the target nucleic acid is a microRNA. In certain embodiments, the target region is in the 5′ UTR of a gene. In certain embodiments, the target region is within a translation suppression element region of a target nucleic acid.

### Certain Compounds

Certain compounds described herein (e.g., antisense agents, oligomeric compounds, and modified oligonucleotides) have one or more asymmetric center and thus give rise to enantiomers, diastereomers, and other stereoisomeric configurations that may be defined, in terms of absolute stereochemistry, as (R) or (S), as a or f such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples are intended to illustrate certain aspects of the invention and are not intended to limit the invention in any way.

### Example 1: Design of RNAi Compounds Targeted to Mouse TTR

Modified oligonucleotides in the table below having stereo-standard nucleosides in the antisense RNAi oligonucleotide and/or the sense RNAi oligonucleotide were synthesized using standard techniques. The antisense RNAi oligonucleotides described in the table below have the sequence TUAUAGAGCAAGAACACUGUUUU (SEQ ID NO: 1) and is complementary to mouse TTR, GenBank Accession No. NM_013697.5 (SEQ ID NO: 13) from nucleoside start site 691 to nucleoside 713. Each antisense RNAi oligonucleotide contains a vinyl phosphonate (vP) moiety at the 5′-position of the 5′-terminal nucleoside.

TABLE 1
Design of antisense RNAi oligonucleotides targeted to mouse TTR

| Compound | Sequence | Sugar Motif | Internucleoside | SEQ ID |
|---|---|---|---|---|
| No. | (5′ to 3′) | (5′ to 3′) | linkages (5′ to 3′) | NO. |
| 1709355 | TUAUAGAGCAAGA | efyfyfyfyfyyyfyfyfy | ssooooooooooooooo | 1 |
| | ACACUGUUUU | fyyy | oooss | |
| 1715030 | TUAUAGAGCAAGA | efyyydyyyyyyyfyfy | ssoooxooooooooooo | 1 |
| | ACACUGUUUU | yyyyyy | oooss | |
| 1715034 | TUAUAGAGCAAGA | efyydfyyyyyyyfyfyy | ssooxoooooooooooo | 1 |
| | ACACUGUUUU | yyyyy | oooss | |

In the table above, a subscript “f” represents a ribo-2′-F sugar, a subscript “y” represents a ribo-2′-OMe sugar, a subscript “e” represents a ribo-2′-MOE sugar, a subscript “d” represents a 2′-β-D-deoxyribosyl sugar, a subscript “s” represents a phosphorothioate internucleoside linkage, a subscript “o” represents a phosphodiester internucleoside linkage, and a subscript “x” represents a methoxypropyl phosphonate internucleoside linkage.

The sense RNAi oligonucleotides are complementary to the first 21 nucleosides of the antisense oligonucleotide (from 5′ to 3′) wherein the last two 3′-nucleosides of the antisense oligonucleotides are not paired with the sense oligonucleotide (are overhanging nucleosides). The sense oligomeric compounds further contain a GalNAc moiety conjugated to the 3′-oxygen as shown below:

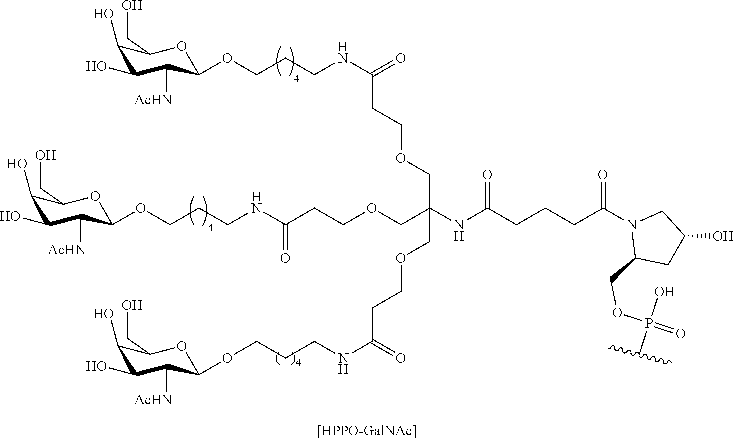

TABLE 2
Design of sense RNAi oligonucleotides targeted to mouse TTR

| Compound | Sequence | Sugar Motif | Internucleoside | SEQ ID |
|---|---|---|---|---|
| No. | (5′ to 3′) | (5′ to 3′) | linkages (5′ to 3′) | NO. |
| 1704053 | AACAGUGUUCUUG | yyyyyyfyfffyyyyyyy | ssooooooooooooooo | 2 |
| | CUCUAUAA | yyy | oo | |
| 1709353 | AACAGUGUUCUUG | fyfyfyfyfffyfyfyfyfy | SSooooooooooooooo | 2 |
| | CUCUAUAA | f | ooo | |

In the table above, a subscript “F” represents a ribo-2′-F sugar, a subscript “y” represents a ribo-2′-OMe sugar, a subscript “s” represents a phosphorothioate internucleoside linkage, and a subscript “o” represents a phosphodiester internucleoside linkage.

TABLE 3
Design of RNAi compounds targeted to mouse TTR

| Duplex | | |
|---|---|---|
| Compound No. | Antisense Compound No. | Sense Compound No. |
| 1709673 | 1709355 | 1709353 |
| 1715049 | 1715034 | 1704053 |
| 1715157 | 1715030 | 1704053 |

### Example 2: Effect of RNAi Duplexes Containing Methoxypropyl Phosphonate Internucleoside Linkages on On-Target Vs Off-Target Activity

The psiCHECK™ reporter vector (Promega) and Dual-Glo® Luciferase Assay System (Promega) were used to compare the effect of RNAi duplexes described herein above on on-target vs off-target activity.

The on-target reporter vector contained a single fully complementary site to the antisense strand of TTR siRNA consisting of the sequence (from 5′ to 3′) AAAACAGTGTTCTTGCTCTATAA (SEQ ID NO: 3) inserted into the 3′-untranslated region of the Renilla luciferase cassette. The off-target reporter vector contained four seed-complementary sites consisting of the sequence (from 5′ to 3′) GCTCTATAA separated by a 19-nucleotide spacer sequence (from 5′ to 3′) TAATATTACATAAATAAAA (SEQ ID NO: 4) inserted into the 3′ untranslated region of the Renilla cassette.

Cos7 cells (ATCC, Manassas, V A) were grown to near confluence before trypsinization, siRNA duplexes (described herein above) and psiCHCECK2 plasmids (Promega) that contain either the on-target or off-target sequences (described herein above) were co-transfected by adding siRNA duplexes at concentrations indicated in the table below together with 5 μl (10 ng) of psiCHECK2 plasmid per well along with 5 μl of Opti-MEM that had been premixed with Lipofectamine 2000 (2 pig/ml) and then incubated at room temperature for 15 min. The mixture was then added to the cells which had been cultured overnight in 100 μl complete media.

48 hours post transfection, levels of Firefly luciferase (transfection control) and Renilla luciferase (fused either to on-target or off-target sequence) were measured per manufacturer protocol. siRNA activity was determined by normalizing the Renilla signal to the Firefly (control) signal within each well. The magnitude of siRNA activity was then assessed relative to cells that had been transfected with psiCHECK2 plasmid in the absence of siRNA (% control). IC50s were calculated using Prism software under a 4 parameter non-linear dose response function.

TABLE 4
Effect of RNAi duplexes containing methoxypropyl phosphonate
internucleoside linkages on on-target vs off-target activity

| | | On- | | | Off- | |
|---|---|---|---|---|---|---|
| | | target | | | target | |
| Com- | Concen- | % | | Concen- | % | |
| pound | tration | con- | IC50 | tration | con- | IC50 |
| No. | (pM) | trol | (pM) | (pM) | trol | (pM) |
| 1709673 | 2000 | 5 | 0.3 | 50000 | 8 | 9 |
| | 400 | 7 | | 10000 | 11 | |
| | 80 | 10 | | 2000 | 14 | |
| | 16 | 18 | | 400 | 24 | |
| | 3.2 | 22 | | 80 | 41 | |
| | 0.64 | 52 | | 16 | 52 | |
| | 0.128 | 66 | | 3.2 | 59 | |
| | 0.0256 | 67 | | 0.64 | 62 | |
| | 0.0051 | 81 | | 0.128 | 77 | |
| | 0.0010 | 90 | | 0.0256 | 86 | |
| | 0.00021 | 106 | | 0.0051 | 105 | |
| | 0.00004 | 105 | | 0.0010 | 109 | |
| 1715049 | 2000 | 5 | 0.2 | 50000 | 10 | 23 |
| | 400 | 8 | | 10000 | 11 | |
| | 80 | 14 | | 2000 | 16 | |
| | 16 | 22 | | 400 | 27 | |
| | 3.2 | 24 | | 80 | 43 | |
| | 0.64 | 43 | | 16 | 60 | |
| | 0.128 | 61 | | 3.2 | 65 | |
| | 0.0256 | 65 | | 0.64 | 73 | |
| | 0.0051 | 83 | | 0.128 | 83 | |
| | 0.0010 | 87 | | 0.0256 | 89 | |
| | 0.00021 | 109 | | 0.0051 | 102 | |
| | 0.00004 | 104 | | 0.0010 | 109 | |
| 1715157 | 2000 | 8 | 0.2 | 50000 | 48 | 27420 |
| | 400 | 8 | | 10000 | 61 | |
| | 80 | 13 | | 2000 | 67 | |
| | 16 | 22 | | 400 | 73 | |
| | 3.2 | 22 | | 80 | 88 | |
| | 0.64 | 43 | | 16 | 90 | |
| | 0.128 | 65 | | 3.2 | 93 | |
| | 0.0256 | 65 | | 0.64 | 89 | |
| | 0.0051 | 80 | | 0.128 | 93 | |
| | 0.0010 | 88 | | 0.0256 | 99 | |
| | 0.00021 | 100 | | 0.0051 | 104 | |
| | 0.00004 | 111 | | 0.0010 | 97 | |

### Example 3: Synthesis of Nucleotide Intermediates with Alkyl Phosphonate Internucleoside Linkages

Nucleotide intermediates containing diisopropylamino alkyl phosphonates were synthesized using the following procedures.

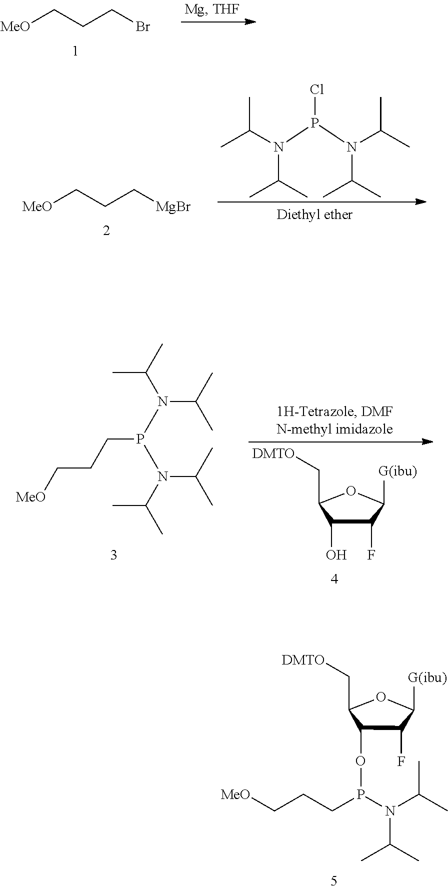

### Synthesis of N,N,N′,N′-tetraisopropyl-1-(3-methoxypropyl)phosphanediamine (3)

Magnesium metal (0.873 g, 35.94 mmol) was suspended in dry THF (30 mL) in a dry round bottom flask and purged with nitrogen. 1-Bromo-3-methoxypropane (Compound 1, 5 g, 2.4 mL, 32.68 mmol) was added in portions via syringe over 20 min. The reaction mixture was slightly exothermic and was immersed in a water bath to control the temperature. The reaction mixture was stirred for an additional 10 min after addition was complete.

In a separate round bottom flask (250 mL), 1-chloro-N,N,N′,N′-tetraisopropylphosphanediamine (5 g, 18.78 mmol) was suspended in anhydrous diethyl ether (60 mL), and the reaction mixture was cooled in an ice bath (0° C.) under nitrogen atmosphere. The compound 2 (generated above) was transferred to the suspension of 1-chloro-N,N,N′,N′-tetraisopropylphosphanediamine via cannula. The reaction mixture was allowed to warm to room temperature. After stirring for 1 h at room temperature, the reaction mixture was filtered through a plug of celite. The filtrate was concentrated under reduced pressure and the residue was suspended in anhydrous acetonitrile (40 mL). The acetonitrile solution was extracted with hexanes (50 mL). The hexanes layer was washed with acetonitrile (2×30 mL), filtered and concentrated a under reduced pressure to yield compound 3 as an oil (3.0 g, 52%), which was immediately used in the next step. ³¹P NMR (121 MHz, CDCl₃) δ: 47.32.

### Synthesis of 5′-O-(4,4′-Dimethoxytrityl)-3′-O-[3-methoxypropyl (diisopropylamino)phosphinyl]-2′-fluorodeoxy-(2-N-isobutyryl)-guanosine (5)

To a solution of compound 4 (2.0 g, 3.04 mmol) in anhydrous DMF (3 mL) were added 1-H-tetrazole (0.17 g, 2.43 mmol) and 1-methyl imidazole (0.11 g, 1.34 mol). Freshly prepared compound 3 (1.39 g, 4.56 mmol) was added, and the reaction mixture was allowed to stir at room temperature for 12-16 h under nitrogen atmosphere. The solvent was removed using a rotary evaporator (maintaining bath temperature under 35° C.) under reduced pressure. The residue obtained was diluted with ethyl acetate (100 mL) and washed with H₂O (200 mL), saturated aqueous NaHCO₃ (2×200 mL), and saturated aqueous NaCl (100 mL). The organic phase dried over was dried over MgSO₄, filtered, and concentrated under reduced pressure. The residue was purified by silica gel column chromatography and eluted with ethyl acetate in hexanes to yield compound 5 (1.3 g, 50%). ³¹P NMR (121 MHz, CDCl₃) δ: 134.2, 134.3 (d), 131.4 (d). ESI-MS m/z: [M−H]⁻ Calcd 859.4, found 859.4.

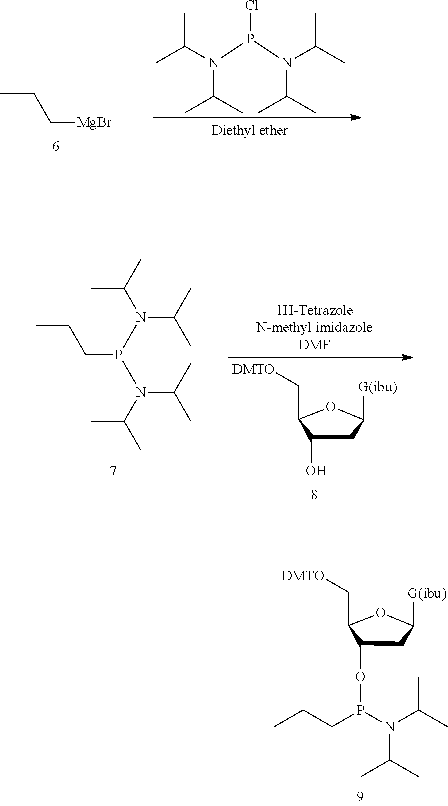

### Synthesis of N,N,N′,N′-tetraisopropyl-1-(3-propyl) phosphanediamine (7)

Compound 6 was prepared from magnesium metal (0.790 g, 32.52 mmol) and bromo-propane (1.85 ml, 20.33 mmol) following the procedure for preparation of alkyl Grignard reagent described above.

Compound 7 was prepared from Compound 6 and 1-chloro-N,N,N′,N′-tetraisopropylphosphanediamine (5 g, 18.78 mmol) in the same manner as compound 3, yielding Compound 7 (3 g, 60%) as an oil which was used immediately in the next reaction. ³¹P NMR (121 MHz, CDCl₃) δ: 47.09.

### Synthesis of 5′-O-(4,4′-Dimethoxytrityl)-3′-O-[propyl(diisopropylamino)phosphinyl]-2′-deoxy-(2-N-isobutyryl)-guanosine (9)

Compound 9 was prepared from compound 8 (1.0 g, 1.56 mmol), 1-H-tetrazole (0.09 g, 1.25 mmol), 1-methyl imidazole (0.06 mg, 0.69 mmol), and compound 7 (0.61 g, 2.34 mmol) in the same manner as compound 5, yielding 9 as a foam (0.9 g, 70%). ³¹P NMR (121 MHz, CDCl₃) δ: 126.6, 125.6. ESI-MS m/z: [M-H]⁻ Calcd 811.4, found 811.4.

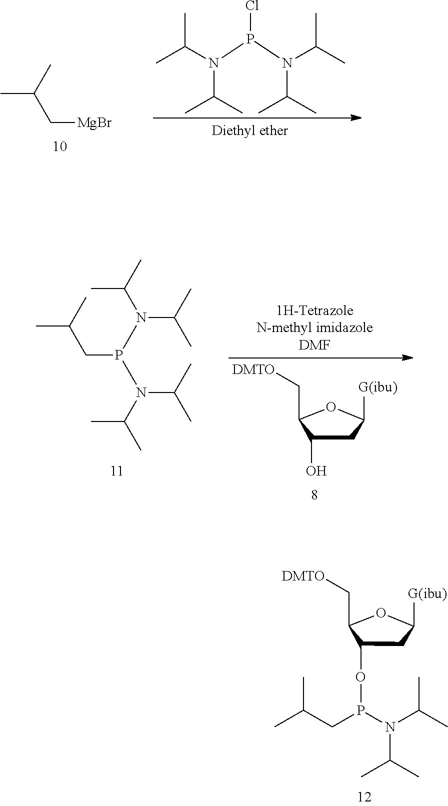

### Synthesis of N,N,N′,N′-tetraisopropyl-1-(isobutyl) phosphanediamine (11)

Compound 11 was prepared from isobutyl magnesium bromide (Compound 10, 3.28 g, 20.33 mmol) and 1-chloro-N,N,N′,N′-tetraisopropylphosphanediamine (5 g, 18.78 mmol) in the same manner as compound 3, yielding 11 (3 g, 93%) as an oil which was used immediately in the next reaction. ³¹P NMR (121 MHz, CDCl₃) δ: 44.43.

### Synthesis of 5′-O-(4,4′-Dimethoxytrityl)-3′-O-[isobutyl(diisopropylamino)phosphinyl]-2′-deoxy-(2-N-isobutyryl)-guanosine (12)

Compound 12 was prepared from compound 8 (0.5 g, 0.781 mmol), 1-H-tetrazole (0.04 g, 0.625 mmol), 1-methyl imidazole (0.03 g, 0.34 mol), and compound 11 (0.338 g, 2.34 mmol) in the same manner as compound 7, yielding 12 (0.9 g, 70%) as a foam. ³¹P NMR (121 MHz, CDCl₃) δ: 125.5, 126.5 (d). ESI-MS m/z: [M-H]⁻ Calcd 825.9, found 825.9.

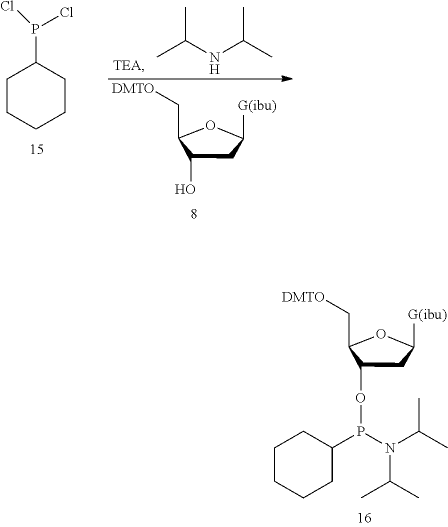

### Synthesis of 5′-O-(4,4′-Dimethoxytrityl)-3′-O-[cyclohexyl(diisopropylamino)phosphinyl]-2′-deoxy-(2-N-isobutyryl)-guanosine (16)

To a solution of N,N-diisopropylamine (0.38 g, 3.75 mmol) in anhydrous acetonitrile (10 mL) at −20° C. was added dichloro(cyclohexyl)phosphane (Compound 15, 0.32 g, 1.72 mmol) and stirred at −20° C. for 40 min. Compound 8 (1 g, 1.56 mmol) in anhydrous dichloromethane (10 mL) and triethylamine (0.22 g, 2.19 mmol) were added and stirred at room temperature overnight under nitrogen. The reaction mixture was concentrated, and residue was purified by silica gel column chromatography and eluted with hexanes/ethyl acetate (1:1) to yield compound 16 (0.25 g, 18%). ³¹P NMR (121 MHz, CDCl₃) δ: 131.7, 132.6 (d). ESI-MS m/z: [M-H]⁻ Calcd 851.4, found 851.4.

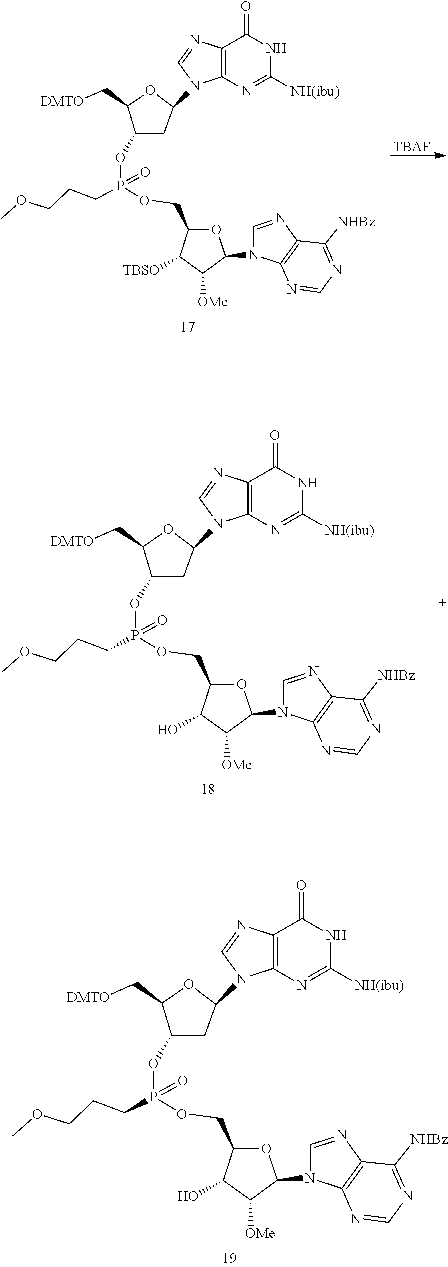

### Synthesis of MOP Sp (18) and Rp (19) dinucleotides

To dinucleotide 17 (Rp/Sp) (0.6 g, 0.48 mmol) TBAF in THF (1 M, 10 mL) was added and the reaction mixture was stirred at room temperature for 12 h. The reaction mixture was concentrated to dryness under reduced pressure and co-evaporated with toluene (2×3 mL). The residue was purified by silica gel column chromatography and eluted with 10% methanol in dichloromethane to afford compound 18 (Sp isomer, 0.14 g) and compound 19 (Rp isomer, 0.28 g). ³¹P NMR (121 MHz, CDCl₃) δ: 34.11 (S). ³¹P NMR (121 MHz, CDCl₃) δ:34.84 (Rp). ESI-MS m/z: [M-H]—1141.3, found 1141.3.

### Example 4: Design of RNAi Compounds Targeted to Mouse TTR

Modified oligonucleotides in the table below having stereo-standard nucleosides in the antisense RNAi oligonucleotide and/or the sense RNAi oligonucleotide were synthesized using standard techniques. The antisense RNAi oligonucleotides described in the table below have the sequence TUAUAGAGCAAGAACACUGUUUU (SEQ ID NO: 1) and is complementary to mouse TTR, GenBank Accession No. NM_013697.5 (SEQ ID NO: 13) from nucleoside start site 691 to nucleoside 713. Each antisense RNAi oligonucleotide contains a vinyl phosphonate (vP) moiety on the 5′-end.

TABLE 5
Design of antisense RNAi oligonucleotides targeted to mouse TTR

| Compound | Sequence | Sugar Motif | Internucleoside | SEQ ID |
|---|---|---|---|---|
| No. | (5′ to 3′) | (5′ to 3′) | linkages (5′ to 3′) | NO. |
| 1708610 | TUAUAGAGCAAGAA | efyyyfyyyyyyyfyfyyy | ssoooooooooooooooo | 1 |
| | CACUGUUUU | yyyy | ooss | |
| 1730877 | TUAUAGAGCAAGAA | efyyydyyyyyyyfyfyy | ssooo[prp]ooooooooo | 1 |
| | CACUGUUUU | yyyyy | oooooss | |
| 1734343 | TUAUAGAGCAAGAA | efyyydyyyyyyyfyfyy | ssooo[ibup]oooooooo | 1 |
| | CACUGUUUU | yyyyy | ooooooss | |
| 1735501 | TUAUAGAGCAAGAA | efyyydyyyyyyyfyfyy | ssooo[chxp]ooooooo | 1 |
| | CACUGUUUU | yyyyy | oooooooss | |

In the table above, “f” represents a ribo-2′-F sugar, “y” represents a ribo-2′-OMe sugar, “e” represents a ribo-2′-MOE sugar, “d” represents a 2′-β-D-deoxyribosyl sugar, “s” represents a phosphorothioate internucleoside linkage, “o” represents a phosphodiester internucleoside linkage, “[prp]” represents a propyl phosphonate internucleoside linkage, “[ibup]” represents a isobutyl phosphonate internucleoside linkage, and “[chxp]” represents a cyclohexyl phosphonate internucleoside linkage.

The sense RNAi oligonucleotide is complementary to the first 21 nucleosides of the antisense oligonucleotide (from 5′ to 3′) wherein the last two 3′-nucleosides of the antisense oligonucleotides are not paired with the sense oligonucleotide (are overhanging nucleosides). The sense oligomeric compound further contains a GalNAc moiety conjugated to the 3′-most oxygen:

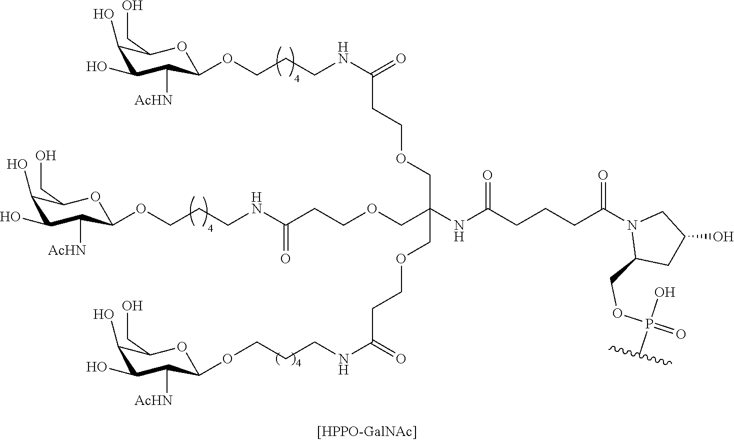

TABLE 6
Design of sense RNAi oligonucleotides targeted to mouse TTR

| Compound | Sequence | Sugar Motif | Internucleoside | SEQ ID |
|---|---|---|---|---|
| No. | (5′ to 3′) | (5′ to 3′) | linkages (5′ to 3′) | NO. |
| 1708605 | AACAGUGUUCUUG | yyyyyyfyfffyyyyyyy | ssoooooooooooooooooo | 2 |
| | CUCUAUAA | yyy | | |

In the table above, “f” represents a ribo-2′-F sugar, “y” represents a ribo-2′-OMe sugar, “s” represents a phosphorothioate internucleoside linkage, and “o” represents a phosphodiester internucleoside linkage.

TABLE 7
Design of RNAi compounds targeted to mouse TTR

| Duplex | | |
|---|---|---|
| Compound No. | Antisense Compound No. | Sense Compound No. |
| 1708616 | 1708610 | 1708605 |
| 1735508 | 1730877 | 1708605 |
| 1735511 | 1734343 | 1708605 |
| 1735514 | 1735501 | 1708605 |

### Example 5: Effect of RNAi Duplexes Containing Alkyl Phosphonate Internucleoside Linkages on On-Target Vs Off-Target Activity

The psiCHECK™ reporter vector (Promega) and Dual-Glo® Luciferase Assay System (Promega) were used to compare the effect of RNAi duplexes described herein above on on-target vs off-target activity.

The on-target reporter vector contained a single fully complementary site to the antisense strand of TTR siRNA consisting of the sequence (from 5′ to 3′) AAAACAGTGTTCTTGCTCTATAA (SEQ ID NO: 3) inserted into the 3′-untranslated region of the Renilla luciferase cassette. The off-target reporter vector contained four seed-complementary sites consisting of the sequence (from 5′ to 3′) GCTCTATAA separated by a 19-nucleotide spacer sequence (from 5′ to 3′) TAATATTACATAAATAAAA (SEQ ID NO: 4) inserted into the 3′ untranslated region of the Renilla cassette.

Cos7 cells (ATCC, Manassas, VA) were grown to near confluence before trypsinization, siRNA duplexes (described herein above) and psiCHCECK2 plasmids (Promega) that contain either the on-target or off target sequences (described herein above) were co-transfected by adding siRNA duplexes at concentrations indicated in the table below together with 5 μl (10 ng) of psiCHECK2 plasmid per well along with 5 gi of Opti-MEM that had been premixed with Lipofectamine 2000 (2 μg/ml) and then incubated at room temperature for 15 min. The mixture was then added to the cells which had been cultured overnight in 100 μl complete media.

48 hours post transfection, levels of Firefly luciferase (transfection control) and Renilla luciferase (fused either to on-target or off-target sequence) were measured per manufacturer protocol siRNA activity was determined by normalizing the Renilla signal to the Firefly (control) signal within each well. The magnitude of siRNA activity was then assessed relative to cells that had ben transfected with psiCHECK2 plasmid in the absence, of siRNA (% control). IC₅₀s were calculated using Prism software under a 4 parameter non-linear dose response function. “N. D.” indicates the value was not determined. “N.C.” indicates the value was not calculated. Each table represents a separate experiment.

TABLE 8
Effect of RNAi duplexes containing alkyl phosphonate internucleoside
linkages on on-target vs off-target activity

| | | On-Target | | | Off-Target | |
|---|---|---|---|---|---|---|
| | | On- | | | Off- | |
| | | target | | | target | |
| Com- | Concen- | % | | Concen- | % | |
| pound | tration | con- | IC₅₀ | tration | con- | IC₅₀ |
| No. | (pM) | trol | (pM) | (pM) | trol | (pM) |
| 1708616 | 2000 | 7 | 0.17 | 50000 | 7 | 14 |
| | 250 | 10 | | 10000 | 8 | |
| | 31.25 | 15 | | 2000 | 12 | |
| | 3.91 | 26 | | 400 | 20 | |
| | 0.488 | 38 | | 80 | 37 | |
| | 0.061 | 66 | | 16 | 63 | |
| | 0.0076 | 98 | | 3.2 | 68 | |
| | 0.00095 | 93 | | 0.64 | 76 | |
| | 0.00012 | 98 | | 0.128 | 91 | |
| | 0.000015 | 103 | | 0.0256 | 98 | |
| | 0.0000019 | 97 | | 0.0051 | 111 | |
| | 0.0000002 | N.D. | | 0.0010 | 113 | |
| 1715157 | 2000 | 8 | 0.21 | 50000 | 51 | N.C. |
| | 250 | 10 | | 10000 | 62 | |
| | 31.25 | 15 | | 2000 | 71 | |
| | 3.91 | 28 | | 400 | 82 | |
| | 0.488 | 41 | | 80 | 93 | |
| | 0.061 | 68 | | 16 | 98 | |
| | 0.0076 | 99 | | 3.2 | 100 | |
| | 0.00095 | 96 | | 0.64 | 94 | |
| | 0.00012 | 101 | | 0.128 | 95 | |
| | 0.000015 | 102 | | 0.0256 | 100 | |
| | 0.0000019 | 98 | | 0.0051 | 105 | |
| | 0.0000002 | N.D. | | 0.0010 | 94 | |
| 1735508 | 2000 | 9 | 0.22 | 50000 | 30 | 1956 |
| | 250 | 11 | | 10000 | 37 | |
| | 31.25 | 17 | | 2000 | 51 | |
| | 3.91 | 28 | | 400 | 73 | |
| | 0.488 | 42 | | 80 | 87 | |
| | 0.061 | 67 | | 16 | 92 | |
| | 0.0076 | 104 | | 3.2 | 93 | |
| | 0.00095 | 91 | | 0.64 | 96 | |
| | 0.00012 | 98 | | 0.128 | 97 | |
| | 0.000015 | 103 | | 0.0256 | 102 | |
| | 0.0000019 | 97 | | 0.0051 | 102 | |
| | 0.0000002 | N.D. | | 0.0010 | 102 | |
| 1735514 | 2000 | 8 | 0.27 | 50000 | 72 | N.C. |
| | 250 | 11 | | 10000 | 74 | |
| | 31.25 | 19 | | 2000 | 86 | |
| | 3.91 | 36 | | 400 | 95 | |
| | 0.488 | 44 | | 80 | 95 | |
| | 0.061 | 70 | | 16 | 98 | |
| | 0.0076 | 92 | | 3.2 | 96 | |
| | 0.00095 | 91 | | 0.64 | 99 | |
| | 0.00012 | 96 | | 0.128 | 98 | |
| | 0.000015 | 102 | | 0.0256 | 99 | |
| | 0.0000019 | 98 | | 0.0051 | 102 | |
| | 0.0000002 | 115 | | 0.0010 | 96 | |
| 1735511 | 2000 | 12 | 0.36 | 50000 | 54 | N.C. |
| | 250 | 14 | | 10000 | 60 | |
| | 31.25 | 20 | | 2000 | 73 | |
| | 3.91 | 29 | | 400 | 89 | |
| | 0.488 | 47 | | 80 | 95 | |
| | 0.061 | 77 | | 16 | 101 | |
| | 0.0076 | 103 | | 3.2 | 96 | |
| | 0.00095 | 95 | | 0.64 | 98 | |
| | 0.00012 | 97 | | 0.128 | 99 | |
| | 0.000015 | 107 | | 0.0256 | 98 | |
| | 0.0000019 | 93 | | 0.0051 | 102 | |
| | 0.0000002 | N.D. | | 0.0010 | 103 | |

### Example 6: Design of RNAi Compounds Targeted to Mouse TTR

RNAi compounds in the table below having stereo-standard nucleosides in the antisense RNAi oligonucleotide and/or the sense RNAi oligonucleotide were synthesized using standard techniques. The antisense RNAi oligonucleotides described in the table below have the sequence TUAUAGAGCAAGAACACUGUUUU (SEQ ID NO: 1) and is complementary to mouse TTR, GenBank Accession No. NM_013697.5 (SEQ ID NO: 13) from nucleoside start site 691 to nucleoside 713. Each antisense RNAi oligonucleotide contains a vinyl phosphonate (vP) moiety at the 5′-position of the 5′-terminal nucleoside.

TABLE 9
Design of antisense RNAi oligonucleotides targeted to mouse TTR

| Compound | | Internucleoside | SEQ |
|---|---|---|---|
| No. | Sugar Motif (5′ to 3′) | linkages (5′ to 3′) | ID NO. |
| 1757577 | edyyyfyyyyyyyfyfyyyyyyy | sxooooooooooooooooooss | 1 |
| 1757578 | efdyyfyyyyyyyfyfyyyyyyy | ssxoooooooooooooooooss | 1 |
| 1757579 | efydyfyyyyyyyfyfyyyyyyy | ssoxooooooooooooooooss | 1 |
| 1715035 | efyyyfdyyyyyyfyfyyyyyyy | ssooooxoooooooooooooss | 1 |
| 1757580 | efyyyfydyyyyyfyfyyyyyyy | ssoooooxooooooooooooss | 1 |
| 1719185 | efyyyfyyyyyyyfyfyyyyyyy | ssoooxooooooooooooooss | 1 |
| 1788167 | efyyyyyyyyyyyfyfyyyyyyy | ssoooxooooooooooooooss | 1 |

In the table above, “f” represents a ribo-2′-F sugar, “y” represents a ribo-2′-OMe sugar, “e” represents a ribo-2′-MOE sugar, “d” represents a 2′-β-D-deoxyribosyl sugar, “s” represents a phosphorothioate internucleoside linkage, “o” represents a phosphodiester internucleoside linkage, and “x” represents a methoxypropyl phosphonate internucleoside linkage.

The sense RNAi oligonucleotides are complementary to the first 21 nucleosides of the antisense oligonucleotide (from 5′ to 3′) wherein the last two 3′-nucleosides of the antisense oligonucleotides are not paired with the sense oligonucleotide (are overhanging nucleosides). Compound No. 1708605 is described herein above.

TABLE 10
Design of RNAi compounds targeted to mouse TTR

| Duplex | | |
|---|---|---|
| Compound No. | Antisense Compound No. | Sense Compound No. |
| 1757587 | 1757577 | 1708605 |
| 1757590 | 1757578 | 1708605 |
| 1757593 | 1757579 | 1708605 |
| 1715052 | 1715035 | 1708605 |
| 1757596 | 1757580 | 1708605 |
| 1719926 | 1719185 | 1708605 |
| 1791379 | 1788167 | 1708605 |

### Example 7: Effect of RNAi Duplexes Containing Alkyl Phosphonate Internucleoside Linkages on On-Target Vs Off-Target Activity

The psiCHECK™ reporter vector (Promega) and Dua-Glo®) Luciferase Assay System (Promega) were used to compare the effect of RNAi duplexes described herein above on on-target vs off-target activity. Cos7 cells (ATCC, Manassas, VA) were grown to near confluence before trypsinization. siRNA duplexes (described herein above) and psiCHCECK2 plasm ids (Promega) that contain either the on-target or off-target sequences (described herein above) were co-transfected by adding RNA duplexes at concentrations indicated in the table below together with 5 μl (10 ng) of psiCHECK2 plasmid per well along with 5 μl of Opti-MEM that had been premixed with Lipofectamine 2000 (2 μg/ml) and then Incubated a room temperature for 15 min. The mixture, was then added to the cells which had been cultured overnight in 100 μl complete media.

48 hours post transfection, levels of Firefly luciferase (transfection control) and Renilla luciferase (fused either to on-target or off-target sequence) were measured per manufacturer protocol. siRNA activity, was determined by normalizing the Renilla signal to the Firefly (control) signal within each well. The magnitude of siRNA activity was then assessed relative to cells that, had been transfected with psiCHECK2 plasmid in the absence of siRNA (% control). IC₅₀ values were calculated using Prism software under a 4 parameter non-linear dose response function. “N.C.” indicates the value was not calculated.

TABLE 11
On-target vs off-target activity of
RNAi duplexes targeted to mouse TTR

| | | On-Target | | | Off-Target | |
|---|---|---|---|---|---|---|
| | | On- | | | Off- | |
| | | target | | | target | |
| Com- | Concen- | % | | Concen- | % | |
| pound | tration | con- | IC₅₀ | tration | con- | IC₅₀ |
| No. | (pM) | trol | (pM) | (pM) | trol | (pM) |
| 1708616 | 2000 | 27 | 3.6 | 50000 | 11 | 22 |
| | 286 | 33 | | 10000 | 23 | |
| | 41 | 42 | | 2000 | 22 | |
| | 5.83 | 41 | | 400 | 27 | |
| | 0.83 | 54 | | 80 | 34 | |
| | 0.12 | 64 | | 16 | 43 | |
| | 0.017 | 76 | | 3.2 | 54 | |
| | 0.0024 | 77 | | 0.64 | 64 | |
| | 0.00035 | 82 | | 0.128 | 76 | |
| | 0.000049 | 93 | | 0.0256 | 105 | |
| | 0.0000071 | 107 | | 0.00512 | 101 | |
| | — | — | | 0.00102 | 95 | |
| 1757587 | 2000 | 37 | 11 | 50000 | 19 | 417 |
| | 286 | 44 | | 10000 | 41 | |
| | 41 | 44 | | 2000 | 37 | |
| | 5.83 | 44 | | 400 | 43 | |
| | 0.83 | 61 | | 80 | 52 | |
| | 0.12 | 67 | | 16 | 64 | |
| | 0.017 | 74 | | 3.2 | 71 | |
| | 0.0024 | 77 | | 0.64 | 84 | |
| | 0.00035 | 87 | | 0.128 | 91 | |
| | 0.000049 | 89 | | 0.0256 | 97 | |
| | 0.0000071 | 111 | | 0.00512 | 99 | |
| | — | — | | 0.00102 | 103 | |
| 1757590 | 2000 | 16 | 3.2 | 50000 | 27 | 29 |
| | 286 | 20 | | 10000 | 35 | |
| | 41 | 25 | | 2000 | 19 | |
| | 5.83 | 34 | | 400 | 26 | |
| | 0.83 | 58 | | 80 | 34 | |
| | 0.12 | 69 | | 16 | 45 | |
| | 0.017 | 82 | | 3.2 | 55 | |
| | 0.0024 | 81 | | 0.64 | 68 | |
| | 0.00035 | 92 | | 0.128 | 84 | |
| | 0.000049 | 94 | | 0.0256 | 94 | |
| | 0.0000071 | 106 | | 0.00512 | 95 | |
| | — | — | | 0.00102 | 111 | |
| 1757593 | 2000 | 9 | 0.81 | 50000 | 20 | 1115 |
| | 286 | 12 | | 10000 | 29 | |
| | 41 | 20 | | 2000 | 30 | |
| | 5.83 | 29 | | 400 | 45 | |
| | 0.83 | 51 | | 80 | 68 | |
| | 0.12 | 61 | | 16 | 79 | |
| | 0.017 | 76 | | 3.2 | 87 | |
| | 0.0024 | 71 | | 0.64 | 84 | |
| | 0.00035 | 79 | | 0.128 | 91 | |
| | 0.000049 | 92 | | 0.0256 | 89 | |
| | 0.0000071 | 108 | | 0.00512 | 105 | |
| | — | — | | 0.00102 | 107 | |
| 1715049 | 2000 | 25 | 2.7 | 50000 | 17 | 157 |
| | 286 | 29 | | 10000 | 31 | |
| | 41 | 32 | | 2000 | 33 | |
| | 5.83 | 38 | | 400 | 45 | |
| | 0.83 | 52 | | 80 | 49 | |
| | 0.12 | 65 | | 16 | 59 | |
| | 0.017 | 83 | | 3.2 | 61 | |
| | 0.0024 | 76 | | 0.64 | 75 | |
| | 0.00035 | 86 | | 0.128 | 81 | |
| | 0.000049 | 92 | | 0.0256 | 103 | |
| | 0.0000071 | 108 | | 0.00512 | 102 | |
| | — | — | | 0.00102 | 96 | |
| 1715157 | 2000 | 34 | 2.9 | 50000 | 54 | N.C. |
| | 286 | 36 | | 10000 | 78 | |
| | 41 | 37 | | 2000 | 77 | |
| | 5.83 | 36 | | 400 | 82 | |
| | 0.83 | 50 | | 80 | 94 | |
| | 0.12 | 64 | | 16 | 92 | |
| | 0.017 | 76 | | 3.2 | 98 | |
| | 0.0024 | 79 | | 0.64 | 98 | |
| | 0.00035 | 86 | | 0.128 | 94 | |
| | 0.000049 | 91 | | 0.0256 | 93 | |
| | 0.0000071 | 109 | | 0.00512 | 101 | |
| | — | — | | 0.00102 | 106 | |
| 1715052 | 2000 | 12 | 0.83 | 50000 | 25 | 118 |
| | 286 | 15 | | 10000 | 43 | |
| | 41 | 20 | | 2000 | 30 | |
| | 5.83 | 27 | | 400 | 29 | |
| | 0.83 | 47 | | 80 | 42 | |
| | 0.12 | 60 | | 16 | 56 | |
| | 0.017 | 75 | | 3.2 | 68 | |
| | 0.0024 | 74 | | 0.64 | 74 | |
| | 0.00035 | 86 | | 0.128 | 89 | |
| | 0.000049 | 93 | | 0.0256 | 94 | |
| | 0.0000071 | 107 | | 0.00512 | 104 | |
| | — | — | | 0.00102 | 102 | |
| 1757596 | 2000 | 18 | 0.64 | 50000 | 20 | 322 |
| | 286 | 20 | | 10000 | 39 | |
| | 41 | 23 | | 2000 | 32 | |
| | 5.83 | 29 | | 400 | 41 | |
| | 0.83 | 45 | | 80 | 54 | |
| | 0.12 | 59 | | 16 | 64 | |
| | 0.017 | 74 | | 3.2 | 72 | |
| | 0.0024 | 70 | | 0.64 | 80 | |
| | 0.00035 | 81 | | 0.128 | 85 | |
| | 0.000049 | 88 | | 0.0256 | 93 | |
| | 0.0000071 | 112 | | 0.00512 | 97 | |
| | — | — | | 0.00102 | 110 | |

### Example 8: Design of RNAi Compounds Targeted to Mouse FXII

RNAi compounds in the table below having stereo-standard nucleosides in the antisense RNAi oligonucleotide and/or the sense RNAi oligonucleotide were synthesized using standard techniques. The antisense RNAi oligonucleotides described in the table below have the sequence TAAAGCACUUUAUUGAGUUUCUG (SEQ ID NO: 5) and is complementary to mouse FXII, GenBank Accession No. NM 021489.3 (SEQ ID NO: 14) from nucleoside start site 1933 to nucleoside 1954, with a single mismatch at the 5′ end. Each antisense RNAi oligonucleotide contains a vinyl phosphonate (vP) moiety at the 5′-position of the 5′-terminal nucleoside. Each cytosine residue is non-methylated unless otherwise indicated, 5-methylcytosines are represented in hold underlined italicized font as “C”.

TABLE 12
Design of antisense RNAi oligonucleotides targeted to mouse FXII

| Compound | | Internucleoside linkages | SEQ |
|---|---|---|---|
| No. | Sugar Motif (5′ to 3′) | (5′ to 3′) | ID NO. |
| 1526195 | efyyyfyyyyyyyfyfyyyyyyy | ssooooooooooooooooooss | 5 |
| 1719168 | efyyyfyyyyyyyfyfyyyyyyy | ssoooxooooooooooooooss | 5 |
| 1719169 | efyyydyyyyyyyfyfyyyyyyy | ssoooxooooooooooooooss | 5 |
| 1719877 | efyyydyyyyyyyfyfyyyyyyy | ssoooxooooooooooooooss | 5 |

In the table above, “f” represents a ribo-2′-F sugar, “y” represents a ribo-2′-OMe sugar. “e” represents a ribo-2′-MOE sugar, “d” represents a 2′-R-D-deoxyribosyl sugar, “s” represents a phosphorothioate internucleoside linkage, “o” represents a phosphodiester internucleoside linkage, and “x” represents a methoxypropyl phosphonate internucleoside linkage.

The sense RNAi oligonucleotide is complementary to the first 21 nucleosides of the antisense oligonucleotide (from 5′ to 3′) wherein the last two 3′-nucleosides of the antisense oligonucleotides are not paired with the sense oligonucleotide (are overhanging nucleosides). The sense oligomeric compound further contains a GalNAc moiety [HPPO-GalNAc]conjugated to the 3′-oxygen, the structure of which is as shown herein above.

TABLE 13
Design of sense RNAi oligonucleotides targeted to mouse FXII

| Compound | Sequence | Sugar Motif | Internucleoside | SEQ ID |
|---|---|---|---|---|
| No. | (5′ to 3′) | (5′ to 3′) | linkages (5′ to 3′) | NO. |
| 1523578 | GAAACUCAAUAAA | yyyyyyfyfffyyyyyyy | ssoooooooooooooooooo | 6 |
| | GUGCUUUA | yyy | | |

In the table above, “f” represents a ribo-2′-F sugar, “y” represents a ribo-2′-OMe sugar, “s” represents a phosphorothioate internucleoside linkage, and “o” represents a phosphodiester internucleoside linkage.

TABLE 14
Design of RNAi compounds targeted to mouse FXII

| Duplex | | |
|---|---|---|
| Compound No. | Antisense Compound No. | Sense Compound No. |
| 1526196 | 1526195 | 1523578 |
| 1719178 | 1719168 | 1523578 |
| 1719181 | 1719169 | 1523578 |
| 1719880 | 1719877 | 1523578 |

### Example 9: Effect of RNAi Duplexes on Mouse FXII in Wild Type Mice

Wild type C57BL/6 mice (Jackson Laboratory) were used to determine the tolerability of the RNAi duplexes and their effects on mouse FXII

### Treatment

Groups of 4 male C57BL/6 mice each received a single subcutaneous injection of RNAi duplexes at various doses indicated in the table below. One group of 4 mice received a single subcutaneous injection of PBS and served as a control group to which RNAi duplex-treated groups were compared. 7 days post treatment, the mice were euthanized.

### Plasma Chemistry Markers

To evaluate the effect of RNAi duplexes on liver and kidney function, plasma levels of albumin (ALB), alanine aminotransferase (ALT), aspartate aminotransferase (AST), toxalbumin (TBIL), creatinine (CREAT), and blood urea nitrogen (BUN) were measured on the day the mice were sacrificed (day 7) using an automated clinical chemistry analyzer (Hitachi Olympus AU400c, Melville, NY). The results were averaged for each group of mice and are presented in the tables below.

TABLE 15
Plasma chemistry markers in C57BL/6 mice

| Com- | Dose | Plasma clinical chemistry | | | | | |
|---|---|---|---|---|---|---|---|
| pound | (mg/ | ALB | ALT | AST | TBIL | CREAT | BUN |
| No. | kg) | (g/dL) | (U/L) | (U/L) | (mg/dL) | (mg/dL) | (mg/dL) |
| PBS | — | 2.77 | 23 | 53 | 0.13 | 0.09 | 27 |
| 1526196 | 0.06 | 2.86 | 23 | 41 | 0.10 | 0.10 | 26 |
| | 0.13 | 2.61 | 61 | 94 | 0.10 | 0.09 | 23 |
| | 0.25 | 2.82 | 22 | 74 | 0.11 | 0.13 | 25 |
| | 0.5 | 2.88 | 67 | 85 | 0.11 | 0.09 | 27 |
| | 1 | 2.93 | 23 | 40 | 0.11 | 0.11 | 26 |
| 1719172 | 0.06 | 2.92 | 25 | 46 | 0.12 | 0.11 | 28 |
| | 0.13 | 2.86 | 21 | 49 | 0.12 | 0.11 | 22 |
| | 0.25 | 2.83 | 24 | 61 | 0.11 | 0.09 | 24 |
| | 0.5 | 2.92 | 33 | 67 | 0.12 | 0.11 | 29 |
| | 1 | 2.71 | 78 | 134 | 0.12 | 0.10 | 26 |
| 1719178 | 0.06 | 2.76 | 24 | 40 | 0.11 | 0.09 | 20 |
| | 0.13 | 2.97 | 53 | 144 | 0.14 | 0.09 | 24 |
| | 0.25 | 2.78 | 51 | 84 | 0.13 | 0.09 | 22 |
| | 0.5 | 2.93 | 25 | 46 | 0.14 | 0.12 | 24 |
| | 1 | 2.83 | 72 | 107 | 0.16 | 0.09 | 22 |
| 1719181 | 0.06 | 2.85 | 25 | 40 | 0.11 | 0.11 | 24 |
| | 0.13 | 3.01 | 26 | 81 | 0.12 | 0.10 | 25 |
| | 0.25 | 2.92 | 21 | 51 | 0.11 | 0.12 | 23 |
| | 0.5 | 2.83 | 55 | 129 | 0.12 | 0.12 | 25 |
| | 1 | 2.90 | 58 | 88 | 0.13 | 0.12 | 24 |
| 1719880 | 0.06 | 2.86 | 120 | 122 | 0.11 | 0.12 | 27 |
| | 0.13 | 2.80 | 21 | 46 | 0.14 | 0.12 | 23 |
| | 0.25 | 2.80 | 36 | 68 | 0.13 | 0.10 | 24 |
| | 0.5 | 2.86 | 42 | 77 | 0.13 | 0.11 | 25 |
| | 1 | 3.02 | 23 | 77 | 0.15 | 0.11 | 23 |

### RNA Analysis

7 days post treatment, mice were sacrificed and RNA was extracted from mouse liver for real-time RTPCR analysis of FXII RNA expression. Mouse FXII primer probe set RTS2959 (forward sequence CAAAGGAGGGACATGTATCAACAC, designated herein as SEQ ID NO: 7; reverse sequence CTGGCAATGTTTCCCAGTGA, designated herein as SEQ ID NO: 8; probe sequence CCCAATGGGCCACACTGTCTCTGC, designated herein as SEQ ID NO: 9) was used to measure mouse FXII RNA levels. FXII RNA levels were normalized to total RNA content, as measured by RIBOGREEN®. Results are presented as percent FXII RNA, relative to the amount of FXII RNA in PBS treated animals (% control). Half maximal effective dose (ED₅₀) of each modified oligonucleotide was calculated using GraphPad Prism 9 software (GraphPad Software, San Diego, CA).

TABLE 16
Effect of RNAi duplexes on mouse FXII RNA in wild type mice

FXII RNA (%
control)

| Compound | Dose | | ED₅₀ |
|---|---|---|---|
| No. | (mg/kg) | Liver | (mg/kg) |
| PBS | — | 100 | — |
| 1526196 | 0.06 | 51 | 0.08 |
| | 0.13 | 48 | |
| | 0.25 | 31 | |
| | 0.5 | 16 | |
| | 1 | 14 | |
| 1719172 | 0.06 | 97 | 1.40 |
| | 0.13 | 99 | |
| | 0.25 | 102 | |
| | 0.5 | 91 | |
| | 1 | 70 | |
| 1719178 | 0.06 | 89 | 0.46 |
| | 0.13 | 78 | |
| | 0.25 | 67 | |
| | 0.5 | 44 | |
| | 1 | 34 | |
| 1719181 | 0.06 | 109 | 0.63 |
| | 0.13 | 92 | |
| | 0.25 | 83 | |
| | 0.5 | 59 | |
| | 1 | 32 | |
| 1719880 | 0.06 | 104 | 1.08 |
| | 0.13 | 112 | |
| | 0.25 | 92 | |
| | 0.5 | 95 | |
| | 1 | 57 | |

### Protein Analysis

Blood plasma was collected on the day the mice were sacrificed (Day 8) for mouse FXII protein analysis. Mouse FXII protein levels were determined using an Innovative Research mouse total Factor XII ELISA kit (IMSFXIIKTT). The results were averaged for each group of mice and are presented in the tables below.

TABLE 17
Levels of mouse FXII Protein in wild type mice

| | | FXII |
|---|---|---|
| Compound | Dose | Protein |
| No. | (mg/kg) | (μg/ml) |
| PBS | — | 24 |
| 1526196 | 0.06 | 12 |
| | 0.13 | 9 |
| | 0.25 | 5 |
| | 0.5 | 2 |
| | 1 | 0.4 |
| 1719172 | 0.06 | 23 |
| | 0.13 | 22 |
| | 0.25 | 19 |
| | 0.5 | 19 |
| | 1 | 16 |
| 1719178 | 0.06 | 22 |
| | 0.13 | 17 |
| | 0.25 | 13 |
| | 0.5 | 11 |
| | 1 | 4 |
| 1719181 | 0.06 | 21 |
| | 0.13 | 20 |
| | 0.25 | 12 |
| | 0.5 | 11 |
| | 1 | 7 |
| 1719880 | 0.06 | 13 |
| | 0.13 | 25 |
| | 0.25 | 17 |
| | 0.5 | 10 |
| | 1 | 23 |

### Example 10: Duration of Action Study of FXII Targeting RNAi Duplexes in Wild Type Mice

The RNAi duplexes described above were tested in wild type C57BL/6 mice (Jackson Laboratory).

### Treatment

Groups of 4 male C57BL/6 mice each received a single subcutaneous injection of RNAi duplexes at a dose of 1 mg/kg. One group of 4 mice received a single subcutaneous injection of PBS and served as a control group to which RNAi duplex-treated groups were compared.

### Protein Analysis

At the various timepoints indicated in the table below blood plasma was collected from the mice via cheek bleed for mouse FXII protein analysis. Mouse FXII protein levels were determined using an Innovative Research mouse total Factor XII ELISA kit (IMSFXIIKTT). The results were averaged for each group of mice and are presented in the tables below.

TABLE 18
Levels of mouse FXII Protein in wild type mice

| | Timepoint | FXII |
|---|---|---|
| Compound | (Days | RNA |
| No. | post-dose) | (μg/ml) |
| PBS | 0 | 28 |
| | 14 | 29 |
| | 28 | 31‡ |
| | 56 | 28‡ |
| | 84 | 26‡ |
| 1526196 | 0 | 22 |
| | 14 | 1 |
| | 28 | 1 |
| | 56 | 6 |
| | 84 | 13 |
| 1719172 | 0 | 24 |
| | 14 | 10 |
| | 28 | 24 |
| | 56 | 25 |
| | 84 | 20 |
| 1719178 | 0 | 27 |
| | 14 | 4 |
| | 28 | 8 |
| | 56 | 19 |
| | 84 | 24 |
| 1719181 | 0 | 23 |
| | 14 | 3 |
| | 28 | 8 |
| | 56 | 22 |
| | 84 | 24 |
| 1719880 | 0 | 28 |
| | 14 | 9 |
| | 28 | 13 |
| | 56 | 29 |
| | 84 | 27 |

‡indicates fewer than 4 samples available

### Example 11: Effect of RNAi Duplexes on Mouse TTR in Wild Type Mice

Wild type C57BL/6 mice (Jackson Laboratory) were used to determine the effect of RNAi duplexes Targeting TTR in wildtype mice.

### Treatment

Groups of 4 male C57BL/6 mice each received a single subcutaneous injection of RNAi duplexes at various doses indicated in the table below. One group of 4 mice received a single subcutaneous injection of PBS and served as a control group to which RNAi duplex-treated groups were compared. 7 days post treatment, the mice were euthanized.

### Plasma Chemistry Markers

To evaluate the effect of RNAi duplexes on liver and kidney function, plasma levels of albumin (ALB), alanine aminotransferase (ALT), aspartate aminotransferase (AST), total bilirubin (TBIL), creatinine (CREAT), blood urea nitrogen (BUN), and glutamate dehydrogenase (GLDH) were measured on the day the mice were sacrificed (day 8) using an automated clinical chemistry analyzer (Hitachi Olympus AU400c, Melville, NY). The results were averaged for each group of mice and are presented in the tables below.

TABLE 19
Plasma chemistry markers in C57BL/6 mice

| | | Plasma clinical chemistry | | | | | | |
|---|---|---|---|---|---|---|---|---|
| Com- | Dose | ALB | ALT | AST | TBIL | CREAT | BUN | GLDH |
| pound | (mg/ | (g/ | (U/ | (U/ | (mg/ | (mg/ | (mg/ | (mmol/ |
| No. | kg) | dL) | L) | L) | dL) | dL) | dL) | L) |
| PBS | — | 2.78 | 30 | 52 | 0.14 | 0.10 | 21 | 17 |
| 1708616 | 0.1 | 2.88 | 23 | 51 | 0.15 | 0.10 | 20 | 15 |
| | 0.3 | 2.74 | 120 | 118 | 0.14 | 0.11 | 22 | 55 |
| | 1 | 2.71 | 117 | 130 | 0.11 | 0.13 | 23 | 62 |
| 1715157 | 0.1 | 2.82 | 18 | 64 | 0.12 | 0.12 | 22 | 13 |
| | 0.3 | 2.83 | 23 | 59 | 0.12 | 0.13 | 24 | 18 |
| | 1 | 2.75 | 73 | 104 | 0.32 | 0.09 | 18 | 39 |
| 1735511 | 0.1 | 3.00 | 60 | 85 | 0.13 | 0.09 | 20 | 46 |
| | 0.3 | 2.74 | 52 | 97 | 0.11 | 0.08 | 17 | 45 |
| | 1 | 2.83 | 28 | 77 | 0.17 | 0.10 | 19 | 21 |
| 1735514 | 0.1 | 2.78 | 38 | 53 | 0.14 | 0.10 | 24 | 28 |
| | 0.3 | 2.77 | 39 | 78 | 0.13 | 0.12 | 21 | 26 |
| | 1 | 3.02 | 40 | 68 | 0.14 | 0.09 | 21 | 27 |

### Body and Organ Weights

Body weights of the mice were measured on day 1 and the average body weight for each group is presented in the table below. Liver, kidney, and spleen weights were measured on the day the mice were sacrificed (day 8), and the average organ weights for each group are presented in the tables below.

TABLE 20
Body and organ weights (in grams)

| | | |
|---|---|---|
| Compound | Dose | Organ weight (g) on Day 8 |

| No. | (mg/kg) | Liver | Kidney | Spleen |
|---|---|---|---|---|
| PBS | — | 1.05 | 0.24 | 0.08 |
| 1708616 | 0.1 | 0.99 | 0.26 | 0.09 |
| | 0.3 | 1.10 | 0.25 | 0.09 |
| | 1 | 1.14 | 0.25 | 0.08 |
| | 3 | 1.16 | 0.25 | 0.08 |
| 1715157 | 0.1 | 1.15 | 0.24 | 0.08 |
| | 0.3 | 1.22 | 0.25 | 0.08 |
| | 1 | 0.89 | 0.28 | 0.10 |
| | 3 | 1.01 | 0.24 | 0.09 |
| 1735511 | 0.1 | 1.11 | 0.25 | 0.07 |
| | 0.3 | 1.23 | 0.27 | 0.09 |
| | 1 | 0.99 | 0.25 | 0.07 |
| | 3 | 1.14 | 0.25 | 0.07 |
| 1735514 | 0.1 | 1.21 | 0.25 | 0.08 |
| | 0.3 | 1.24 | 0.28 | 0.09 |
| | 1 | 1.12 | 0.25 | 0.08 |
| | 3 | 1.08 | 0.26 | 0.09 |

### RNA Analysis

7 days post treatment, mice were sacrificed and RNA extracted from mouse liver for real-time RTPCR analysis of TTR RNA expression. Mouse TTR primer probe set mTTR_1 (forward sequence CGTACTGGAAGACACTTGGCATT, designated herein as SEQ ID NO: 10; reverse sequence GAGTCGTTGGCTGTGAAAACC, designated herein as SEQ ID NO: 11; probe sequence CCCGTTCCATGAATTCGCGGATG, designated herein as SEQ ID NO: 12) was used to measure mouse TTR RNA levels. TTR RNA levels were normalized to total RNA content, as measured by RIBOGREEN®. Results are presented as percent TTR RNA, relative to the amount of TTR in PBS treated animals (% control). Half maximal effective dose (ED₅₀) of each modified oligonucleotide was calculated using GraphPad Prism 9 software (GraphPad Software, San Diego, CA).

TABLE 21
Effect of RNAi duplexes on mouse TTR RNA in wild type mice

TTR RNA (%
control)

| Compound | Dose | | ED50 |
|---|---|---|---|
| No. | (mg/kg) | Liver | (mg/kg) |
| PBS | — | 100 | — |
| 1708616 | 0.1 | 53 | 0.11 |
| | 0.3 | 19 | |
| | 1 | 3 | |
| | 3 | 1 | |
| 1715157 | 0.1 | 89 | 0.36 |
| | 0.3 | 57 | |
| | 1 | 15 | |
| | 3 | 3 | |
| 1735511 | 0.1 | 71 | 0.26 |
| | 0.3 | 52 | |
| | 1 | 12 | |
| | 3 | 2 | |
| 1735514 | 0.1 | 88 | 0.54 |
| | 0.3 | 73 | |
| | 1 | 27 | |
| | 3 | 8 | |

### Example 12: Off-Target Effects of RNAi Duplexes Targeted to TTR in Wild Type Mice

Wild type C57BL/6 mice (Jackson Laboratory) were used to determine the effect of RNAi duplexes described herein above on off-targets.

### Treatment

Groups of 4 male C57BL/6 mice each received a single subcutaneous injection of RNAi duplex compound at doses of 0.3 mg/kg, 1 mg/kg, 3 mg/kg. One group of 4 mice received a single subcutaneous injection of PBS and served as a control group to which RNAi duplex-treated groups were compared. 7 days post treatment, the mice were euthanized.

### DGE Analysis

Liver tissue extracted from the mice was subjected to DGE (Digital Gene Expression) analysis for 3′ end transcriptome profiling using the QuantSeq 3′ mRNA-Seq Library Prep Kit FWD for Illumina on the Illumina sequencing platform.

Digital gene expression analysis resulted in the generation of 4 million unique, mapped reads, with expression data for 10,900 unigenes. A total of 312 unique differentially expressed genes with a fold change greater than 2, a p value less than 0.01 and a q value less than 0.1 were identified between the saline and RNAi duplex treated group (RNAi duplex treated at 3 doses: 0.3 mg/kg, 1.0 mg/kg and 3.0 mg/kg each vs Saline).

The number of differentially expressed genes detected with each RNAi duplex treatment are listed in the table below.

TABLE 22
Number of differentially expressed genes
detected with RNAi duplex treatment

| | |
|---|---|
| Compound | |
| No. | Number of differentially expressed genes detected |

| | |
|---|---|
| 1708616 | 234 |
| 1715157 | 60 |
| 1735511 | 11 |
| 1735514 | 108 |

What is claimed:

1. An RNAi agent comprising an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, wherein the antisense RNAi oligonucleotide comprises at least one region having Formula I: 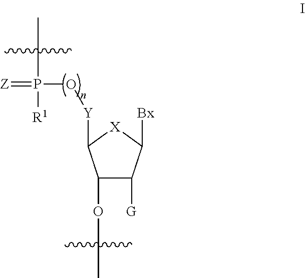
wherein
X is O or O—CH₂;
Z is O or S,
R¹ is selected from OH, C₁-C₂₀ alkyl, C₁-C₂₀ heteroalkyl, C₃-C₁₀ cycloalkyl, C₆-C₁₀ aryl, 3-10 membered heterocyclyl, and 3-10 membered heteroaryl, and wherein R¹ is optionally substituted with C₁-C₂₀ alkyl, C₁-C₂₀ heteroalkyl, C₃-C₁₀ cycloalkyl, C₆-C₁₀ aryl, 3-10 membered heterocyclyl, 3-10 membered heteroaryl, OR^{D}, NR^{D}R^{D}, SR^{D}, or R^{A}COOR^{D}, wherein R^{A} is C₁-C₆ alkyl and each R^{D} is independently H or C₁-C₆ alkyl;
n is 0 or 1;
Y is C₁-C₆ alkyl, C₃-C₁₀ cycloalkyl, or 3-10 membered heterocyclyl; provided that if n is 1 and Y is CH₂ or CH₂(CH₃), then R¹ is not OH;
and
each G is independently H, halogen, OH, or O[C(R⁴)(R⁵)]ₚ—[(C═O)_{q}—R⁶]ⱼ—R⁷; wherein
each R⁴ and R⁵ is, independently, H, halogen, C₁-C₆ alkyl or substituted C₁-C₆ alkyl;
each R⁶ is O, S or N(E¹);
R⁷ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, or N(E²)(E³);
E¹, E² and E³ are each, independently, H, C₁-C₆ alkyl, or substituted C₁-C₆ alkyl;
p is from 1 to 6;
q is 0 or 1;
j is 0 or 1;
each substituted group comprises one or more optionally protected substituent groups independently selected from halogen, OJ¹, N(J¹)(J²), =NJ¹, SJ¹, N₃, CN, OC(=Q)J¹, OC(=Q)-N(J¹)(J²), and C(=Q)N(J¹)(J²);
Q is O, S or NJ³;
J¹, J² and J³ are each, independently, H or C₁-C₆ alkyl;
Bx is a heterocyclic base moiety.

2. The RNAi agent of claim 1, wherein X is O.

3. The RNAi agent of claim 1, wherein X is O—CH₂.

4. The RNAi agent of any of claims 1-3, wherein Z is O.

5. The RNAi agent of any of claims 1-3, wherein Z is S.

6. The RNAi agent of any of claims 1-5, wherein n is 1.

7. The RNAi agent of any of claims 1-6, wherein Y is CH₂.

8. The RNAi agent of any of claims 1-7, wherein R¹ is —[C(R⁸)(H)]ₘO[C(R⁹)(H)]ₜ—R¹⁰, wherein
each R⁸ and R⁹ is independently selected from H or C₁-C₃ alkyl;
R¹⁰ is H or halogen;
m is from 1 to 6;
and t is from 1 to 6.

9. The RNAi agent of claim 8, wherein each R⁸ is H, and m is from 1-3.

10. The RNAi agent of claim 9, wherein each R⁹ is H and t is from 1-3.

11. The RNAi agent of claim 8, wherein R¹ is methoxypropyl.

12. The RNAi agent of any of claims 1-7, wherein R¹ is C₁-C₆ alkyl.

13. The RNAi agent of claim 12, wherein R¹ is propyl or isopropyl.

14. The RNAi agent of claim 12, wherein R¹ is propyl.

15. The RNAi agent of claim 12, wherein R¹ is isobutyl.

16. The RNAi agent of any of claims 1-10, wherein R¹ is cyclohexyl.

17. The RNAi agent of any of claims 1-5, wherein n is 0 and Y is cyclopropyl.

18. The RNAi agent of any of claims 1-17, wherein G is selected from OMe, F, and H.

19. The RNAi agent of claim 18, wherein G is F.

20. The RNAi agent of claim 18, wherein G is OMe.

21. The RNAi agent of claim 18, wherein G is H.

22. The RNAi agent of any of claims 1-21, having exactly one region having Formula I.

23. The RNAi agent of claim 22, wherein the region having Formula I is within the first 8 nucleosides of the antisense RNAi oligonucleotide.

24. The RNAi agent of claim 22, wherein the region having Formula I includes the 6^{th} nucleoside from the 5′ end of the antisense RNAi oligonucleotide.

25. The RNAi agent of claim 22, wherein the region having Formula I includes the 7^{th} nucleoside from the 5′ end of the antisense RNAi oligonucleotide.

26. An RNAi agent, comprising an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, and wherein the antisense RNAi oligonucleotide contains at least one internucleoside linkage having Formula II: 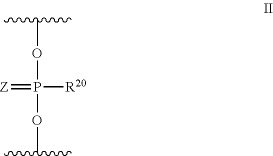
wherein Z is O or S;
R²⁰ is selected from C₁-C₂₀ alkyl, C₁-C₂₀ heteroalkyl, C₃-C₁₀ cycloalkyl, C₆-C₁₀ aryl, 3-10 membered heterocyclyl, and 3-10 membered heteroaryl, and wherein R²⁰ is optionally substituted with C₁-C₂₀ alkyl, C₁-C₂₀ heteroalkyl, C₃-C₁₀ cycloalkyl, C₆-C₁₀ aryl, 3-10 membered heterocyclyl, 3-10 membered heteroaryl, OR^{D}, NR^{D}R^{D}, SR^{D}, or R^{A}COOR^{D}, wherein R^{A} is C₁-C₆ alkyl and each R^{D} is independently H or C₁-C₆ alkyl.

27. The RNAi agent of claim 26, wherein R²⁰ is R²¹; wherein R²¹ is —[C(R⁸)(R⁹)]ₘ-(Q)ₙ[C(R⁸)(R⁹)]ₜ—R¹⁰ wherein;
Q is O, NR^{D}, or S;
each R⁸ is independently selected from H and C₁-C₃ alkyl;
each R⁹ is independently selected from H and C₁-C₃ alkyl;
R¹⁰ is H, halogen, OR^{D}, NR^{D}R^{D}, SR^{D}, or COOR^{D}, wherein each R^{D} is independently H or C₁-C₃ alkyl;
n is 0 or 1;
m is from 1 to 6;
and t is from 0 to 6.

28. The RNAi agent of claim 27, wherein Q is O.

29. The RNAi agent of claim 26 or 27, wherein Z is O.

30. The RNAi agent of any of claims 27-29, wherein n is 1.

31. The RNAi agent of any of claims 26-30, wherein R⁸, R⁹, and R¹⁰ are each H.

32. The RNAi agent of any of claims 26-31, wherein m is 3.

33. The RNAi agent of any of claims 26-32, wherein t is 1.

34. The RNAi agent of any of claims 27-33, wherein n is 0.

35. The RNAi agent of claim 34, wherein R⁸, R⁹, and R¹⁰ are each H.

36. The RNAi agent of claim 34 or 35, wherein m+t equals 3.

37. The RNAi agent of claim 26 or 29, wherein R²⁰ is isobutyl.

38. The RNAi agent of claim 26 or 29, wherein R²⁰ is propyl.

39. The RNAi agent of claim 35, wherein m is 1, t is 0, R⁸ is H and R¹⁰ is COOR^{D}.

40. The RNAi agent of claim 39, wherein R^{D} is H.

41. The RNAi agent of claim 26 or 29, wherein R²⁰ is methoxypropyl.

42. The RNAi agent of claim 26 or 29, wherein R²⁰ is C₃-C₁₀ cycloalkyl.

43. The RNAi agent of claim 26 or 29, wherein R²⁰ is cyclohexyl.

44. The RNAi agent of claim 26 or 29, wherein R²⁰ is not methyl.

45. The RNAi agent of any of claims 26-44, wherein the internucleoside linkage having Formula II is within the seed region of the antisense RNAi oligonucleotide.

46. The RNAi agent of any of claims 26-45, wherein the internucleoside linkage having Formula II is between nucleosides 5 to 6 or nucleosides 6 to 7 of the antisense RNAi oligonucleotide, counting from the 5′-end.

47. The RNAi agent of claim 46, wherein the internucleoside linkage having Formula II is between nucleosides 6 and 7 of the antisense RNAi oligonucleotide, counting from the 5′ end.

48. The RNAi agent of any of claims 26-47, wherein each internucleoside linkage of the antisense RNAi oligonucleotide that does not have Formula II is selected from a phosphorothioate internucleoside linkage and a phosphodiester internucleoside linkage.

49. The RNAi agent of any of claims 26-47, wherein each internucleoside linkage of the antisense RNAi oligonucleotide that does not have Formula II is selected from a phosphorothioate internucleoside linkage and a phosphodiester internucleoside linkage, and a mesyl phosphoramidate internucleoside linkage.

50. The RNAi agent of any of claims 26-47, wherein the antisense RNAi oligonucleotide consists of 23 linked nucleosides and has an internucleoside linkage motif selected from ssooqoooooooooooooooss or ssooogooooooooooooooss, wherein each “s” represents a phosphorothioate internucleoside linkage, each “o” represents a phosphodiester internucleoside linkage, and each “q” represents an internucleoside linkage of Formula II.

51. The RNAi agent of claim 50, wherein each “q” represents a methoxypropyl internucleoside linkage.

52. The RNAi agent of any of claims 1-51, wherein the antisense RNAi oligonucleotide consists of 21 linked nucleosides.

53. The RNAi agent of any of claims 1-51, wherein the antisense RNAi oligonucleotide consists of 23 linked nucleosides.

54. The RNAi agent of any of claims 1-53, wherein the sense RNAi oligonucleotide consists of 19 linked nucleosides.

55. The RNAi agent of any of claims 1-53, wherein the sense RNAi oligonucleotide consists of 21 linked nucleosides.

56. The RNAi agent of any of claims 1-55, wherein the antisense RNAi oligonucleotide comprises a 5′-stabilized phosphate moiety.

57. The RNAi agent of claim 56, wherein the stabilized phosphate moiety is a 5′-vinyl phosphonate, a 5′-methylene phosphonate, or a 5′-cyclopropyl phosphonate.

58. The RNAi agent of any of claims 1-57, wherein the antisense RNAi oligonucleotide does not comprise a GNA or a UNA nucleoside.

59. The RNAi agent of any of claims 1-58, wherein each nucleoside of the antisense RNAi oligonucleotide comprises a sugar moiety selected from 2′-F, 2′-OMe, 2′-deoxy, 2′-MOE, or an FHNA sugar surrogate.

60. The RNAi agent of any of claims 1-59, wherein the 5′-most nucleoside of the antisense RNAi oligonucleotide comprises a 5′-vinylphosphonate-2′-O-methoxyethyl-β-D-ribosyl sugar moiety.

61. The RNAi agent of any of claims 1-60, wherein the nucleoside immediately to the 5′ of the region of Formula I or internucleoside linkage having Formula II comprises a ribo-2′-F or 2′-β-D-deoxyribosyl sugar moiety.

62. The RNAi agent of any of the preceding claims, wherein one, two, or three of the nucleosides at positions 3, 4, 5 from the 5′-terminus of the antisense RNAi oligonucleotide are 2′-OMe nucleosides.

63. The RNAi agent of any of the preceding claims, wherein each of the nucleosides at positions 3, 4, 5 from the 5′-terminus of the antisense RNAi oligonucleotide are 2′-OMe nucleosides.

64. The RNAi agent of any of the preceding claims, wherein one, two, three, four, or five of the nucleosides at positions 7, 8, 9, 10, and 11 from the 5′-terminus of the antisense RNAi oligonucleotide are 2′-OMe nucleosides.

65. The RNAi agent of any of the preceding claims, wherein each of the nucleosides at positions 7, 8, 9, 10, and 11 from the 5′-terminus of the antisense RNAi oligonucleotide are 2′-OMe nucleosides.

66. The RNAi agent of any of the preceding claims, wherein one, two, three, four, or five of the nucleosides at positions 17, 18, 19, 20, and 21 from the 5′-terminus of the antisense RNAi oligonucleotide are 2′-OMe nucleosides.

67. The RNAi agent of any of the preceding claims, wherein each of the nucleosides at positions 17, 18, 19, 20, and 21 from the 5′-terminus of the antisense RNAi oligonucleotide are 2′-OMe nucleosides.

68. The RNAi agent of of any of the preceding claims, wherein at least one nucleoside in the antisense RNAi oligonucleotide is a 2′-F nucleoside.

69. The RNAi agent of any of the preceding claims, wherein one, two, or three of the nucleosides at positions 2, 14, and 16 from the 5′-terminus of the antisense RNAi oligonucleotide are 2′-F nucleosides.

70. The RNAi agent of any of the preceding claims, wherein each of the nucleosides at positions 2, 14, and 16 from the 5′-terminus of the antisense RNAi oligonucleotide are 2′-F nucleosides.

71. The RNAi agent of any of the preceding claims, wherein the nucleoside at position 6 from the 5′-terminus of the antisense RNAi oligonucleotide is a 2′-F nucleoside.

72. The RNAi agent of any of the preceding claims, wherein the nucleoside at position 6 from the 5′-terminus of the antisense RNAi oligonucleotide is a 2′-OMe nucleoside.

73. The RNAi agent of any of the preceding claims, wherein wherein at least one nucleoside in the antisense RNAi oligonucleotide is a 2′-deoxynucleoside.

74. The RNAi agent of any of the preceding claims, wherein one, two, or three of the nucleoside at positions 5, 6, 7 from the 5′-terminus of the antisense RNAi oligonucleotide is a 2′-deoxynucleoside.

75. The RNAi agent of any of the preceding claims, wherein the nucleoside at position 6 from the 5′-terminus of the antisense RNAi oligonucleotide is a 2′-deoxynucleoside.

76. The RNAi agent of any of the preceding claims, wherein one or two of the nucleoside at positions 5 and 7 from the 5′-terminus of the antisense RNAi oligonucleotide is a 2′-deoxynucleoside.

77. The RNAi agent of any of the preceding claims, wherein wherein at least one nucleoside in the antisense RNAi oligonucleotide is a 2′-MOE nucleoside.

78. The RNAi agent of any of the preceding claims, wherein one, two, three, four, or five of the nucleosides at positions 1, 19, 20, 21, 22, and 23 from the 5′-terminus of the antisense RNAi oligonucleotide are 2′-MOE nucleosides.

79. The RNAi agent of any of the preceding claims, wherein the nucleoside at position 1 from the 5′-terminus of the antisense RNAi oligonucleotide is a 2′-MOE nucleoside.

80. The RNAi agent of any of the preceding claims, wherein each of the nucleosides at positions 1, 22, and 23 from the 5′-terminus of the antisense RNAi oligonucleotide are 2′-MOE nucleosides.

81. The RNAi agent of any of the preceding claims, wherein one or two of the nucleosides at positions 9 and 10 from the 5′-terminus of the antisense RNAi oligonucleotide are 2′-MOE nucleosides.

82. The RNAi agent of any of claims 1-25, wherein each internucleoside linkage of the antisense RNAi oligonucleotide other than the region having Formula I is selected from a phosphorothioate internucleoside linkage and a phosphodiester internucleoside linkage.

83. The RNAi agent of any of claims 1-25, wherein each internucleoside linkage of the antisense RNAi oligonucleotide other than the region having Formula I is selected from a phosphorothioate internucleoside linkage, a phosphodiester internucleoside linkage, and a mesyl phosphoramidate internucleoside linkage.

84. A population of RNAi agents of any of claims 1-83, wherein the population is chirally enriched for oligonucleotides having a particular stereochemical configuration at one or more internucleoside linkages.

85. The population of claim 84, wherein the stereochemically enriched internucleoside linkage is in the region of Formula I or the internucleoside linkage of Formula II.

86. The population of claim 85, wherein the internucleoside linkage in the region of Formula I or the internucleoside linkage of Formula II is in the R_{P} configuration.

87. The population of claim 85, wherein the internucleoside linkage in the region of Formula I or the internucleoside linkage of Formula II is in the S_{P} configuration.

88. A population of RNAi agents of any of claims 1-83, wherein the stereochemical configuration at the internucleoside linkage in the region of Formula I or the internucleoside linkage of Formula II is stereorandom.

89. A pharmaceutical composition comprising the RNAi agent or population of any of claims 1-88 and a pharmaceutically acceptable carrier or diluent.

90. A method comprising contacting a cell with the RNAi agent or population of any of claims 1-88 or pharmaceutical composition of claim 89.

91. A method of modulating the amount or activity of a target nucleic acid in a cell, comprising contacting the cell with the RNAi agent of any of claims 1-88 or pharmaceutical composition of claim 89 and thereby modulating the amount or activity of the target nucleic acid.

92. A method of modulating the amount or activity of a target nucleic acid in a cell, comprising contacting the cell with the RNAi agent of any of claims 1-88 or pharmaceutical composition of claim 89.

93. The method of claims 91 or 92, wherein the amount or activity of a target nucleic acid is reduced.

94. Use of the RNAi agent, population, or composition of any of claims 1-89 for treatment of a disease or condition.

95. Use of the RNAi agent, population, or composition of any of claims 1-89 for a preparation of a medicament for treatment of a disease or condition.
